(19) United States
(12) Patent Application Publication
(10) Pub. No.: US 2026/0295117 A1
(43) Pub. Date: Oct. 1, 2026

(54) CROSSLINKABLE LIQUID COMPOSITION FOR USE IN A METHOD OF TREATING OR PREVENTING AN EYE DISORDER

(71) Applicants: Universiteit Antwerpen, Antwerpen (BE); Universitair Ziekenhuis Antwerpen, Edegem (BE)

(72) Inventors: Bert VAN DEN BOGERD, Wilrijk (BE); Sorcha NÍ DHUBHGHAILL, Edegem (BE)

(73) Assignees: Universiteit Antwerpen, Antwerpen (BE); Universitair Ziekenhuis Antwerpen, Edegem (BE)

(21) Appl. No.: 19/478,565

(22) PCT Filed: Apr. 26, 2024
(86) PCT No.: PCT/EP2024/061509

§ 371 Date: Oct. 24, 2025

(30) Foreign Application Priority Data
Apr. 26, 2023 (EP) 23170032.9

(51) Int. Cl. A61L 27/22; A61L 27/50

(52) U.S. Cl. CPC A61L 27/222; A61L 27/50; A61L 2400/06; A61L 2430/16

(57) ABSTRACT

The invention concerns a crosslinkable liquid composition for use in a method of curative or preventive treatment of an eye disorder in an eye of a subject, or for use in a surgical method of preparing a corneal onlay on or a corneal inlay in an eye of a subject, wherein the crosslinkable liquid composition comprises: a crosslinkable biomaterial, wherein the crosslinkable biomaterial comprises a gelatin functionalized with one or more tyrosine-derived or phenol groups; and a visible and/or UV light photoinitiator system.

### FIELD OF THE INVENTION

The invention is broadly in the field of medicine, more precisely in the field of ophthalmology. In particular, the invention concerns the use of a crosslinkable liquid composition in a method of curative or preventive treatment of an eye disorder such as cataract, macular degeneration, a refractive error, or a chronic or subacute corneal disease involving an irregularity of the cornea in a subject.

### BACKGROUND OF THE INVENTION

The cornea is the transparent tissue in the front of the eye. It functions as the window of the eye and is responsible for two thirds of the refraction of the incoming light, while the remaining refraction is done by the crystalline lens. Proper refraction of incoming light is necessary to project a clear image onto the retina, where light is converted to electrical signals that are transmitted via the optic nerve to the visual cortex in the brain. Refractive disorders, such as myopia, hyperopia, astigmatism and presbyopia, can blur vision for various distances and are, in most cases, the result of an aberrant corneal curvature. A lens can be placed in front of the eye as a correction, and that can either be in the shape of contact lenses or spectacles, to compensate the aberrant light path. Uncorrected refractive errors can lead to (severe) visual impairment and, secondarily, to headaches, fatigue and eye irritation. That is why refractive errors are listed as the most prevalent cause of reversible blindness worldwide.

Another solution to correcting refractive disorders is to reshape the cornea by means of refractive laser surgery, termed laser ablation or photoablation, of which different techniques exist including photorefractive keratectomy (PRK), laser assisted in situ keratomileusis (LASIK) and small incision lenticule extraction (SMILE). The common denominator of these procedures is that they aim to adjust the curvature of the cornea by means of ablation, i.e. removing corneal tissue by means of a laser pulse. This tissue dissection will result in change of the corneal curvature and thus correcting the corneal focusing power to properly focus light onto the retina. In the case of hyperopia, the central optical zone is relatively steepened by applying laser pulses to the corneal midperiphery, while in myopia the central cornea is flattened to reduce the curvature.

Photoablative treatment for refractive errors, like PRK, LASIK and SMILE, have limitations as they are all fundamentally subtractive. Since tissue is removed from the cornea to relatively steepen or flatten the curvature, the most obvious limitation is the extent of the tissue removal required to achieve the effect. Removing too much corneal tissue renders a thin cornea that is prone to ectasia (corneal thinning) and corneal perforation. For hyperopia, photo-ablative laser surgery is safe and effective up to +2 dioptres (dpt) in practice, but becomes less predictable in higher degrees. This is also the case for astigmatism up to 3 dpt. Secondly, the effect of refractive surgery (for hyperopia or presbyopia) tends to regress over time since epithelial cells overgrow the induced grooves in the cornea, thereby returning to a state similar to its initial aberrant curvature. Thirdly, refractive surgery has, depending on the specific technique, risks associated as a result of suboptimal procedure such as risk of corneal haze and long visual recovery periods (PRK), flap related problems (LASIK) or complications due to challenging technical methods (SMILE).

Furthermore, the constant exposure of our eyes to ultraviolet (UV) (<400 nm) and high-energy visible (HEV) or blue light (400-500 nm) can damage the eye and has been linked to diseases such as cataract and macular degeneration. The main source of UV and HEV is natural sunlight, but also electronic devices such as computer screens and smart phones emit blue light. Due to our increased use of electronic devices, we are significantly exposed to blue light, which can negatively impact our retina on the long term. Blue HEV light is particularly accountable for light scattering and misfocus—which is aggravated during night time conditions—leading to disability glare, discomfort glare, chromatic contrast, starbursts. At this moment, blue light filters are often recommended for patients to prevent development and progression of, among others, age related macular degeneration, to avoid retinal phototoxicity in general or reduce the side effects of HEV light in general (e.g., during driving or prolonged periods of screen time). Currently UV and blue light filters are frequently found in intraocular lenses (IOL), which are implanted at the time of cataract surgery, or in blue light blocking contact lenses or spectacles. The problem is that an integrated UV and blue light blocking filter of IOL is only possible for individuals undergoing cataract surgery and is irreversible and expensive. Shortcomings of blue light filtering contact lenses are dry eyes and the risk for infection. Disadvantages of blue light blocking spectacles are the same as for normal glasses and contact lenses, namely the risk of losing them and that the patient has to wear them to have the effect.

In view thereof, there remains a need in the art for further and/or improved compositions and methods for protection of the eyes from incoming UV and HEV light and the concomitant treatment of cataract and macular degeneration, while also being useful for treating eye disorders such as refractive disorders.

### SUMMARY OF THE INVENTION

The present inventors have found a crosslinkable liquid composition and its use in a method of curative or preventive treatment of an eye disorder, thereby addressing one or more of the above-mentioned problems in the art.

Accordingly, a first aspect of the invention relates to a crosslinkable liquid composition for use in a method of curative or preventive treatment of an eye disorder in an eye of a subject, wherein the crosslinkable liquid composition comprises:
a crosslinkable biomaterial, wherein the crosslinkable biomaterial comprises a gelatin functionalized with one or more tyrosine-derived or phenol groups; and
a visible and/or UV light photoinitiator system.

The present inventors realized that by choosing a particular crosslinkable liquid composition, e.g., comprising a gelatin functionalized with one or more tyrosine-derived or phenol groups and a visible and/or UV light photoinitiator system, the corneal onlay develops UV and blue light blocking properties, thereby acting as a filter. Furthermore, this crosslinkable liquid composition has the additional advantage that allows crosslinking in a matter of seconds—as opposed to the traditional UV based crosslinking systems—making the risk for side effects and overall treatment time lower. By using this crosslinkable liquid composition, a corneal onlay or corneal inlay can be formed very quickly, providing immediate protection against HEV and UV light. Further, due to the very fast crosslinking kinetics, the crosslinking duration (irradiation time) of the eye is drastically reduced, thereby reducing the risks of side-effects and decreasing the overall treatment time.

Moreover, the present invention is at least in part based on the inventors' innovative insight and experimental evaluation that a refractive error or a chronic or subacute corneal disease or disorder involving an irregularity of the cornea can be treated by applying a crosslinkable composition onto the anterior corneal surface of the affected eye as a liquid using a mold, subsequently crosslinking the composition in situ. It is then possible, when necessary to correct the curvature of the newly formed corneal onlay, using a technique such as photoablation, without subtracting the stroma volume from the cornea per se. The advantages of crosslinking the tissue in situ are that the crosslinking causes intermolecular crosslinking of the liquid composition and that simultaneously the UV or visible light results in crosslinking (or connecting) the composition with the cornea thereby ensuring adhesion to the cornea through chemical interaction. In this way, problems which are common with lenses such as infection are avoided. Additionally, as the crosslinkable biomaterial is liquid, it perfectly fits the patient's corneal geometry compared to prefabricated onlays.

The method of the present invention provides a long-term, but reversible, solution for treating eye disorders, such as a refractive error or a chronic or subacute corneal disease involving an irregularity of the cornea, while at the same time providing protection of the eyes from incoming UV and HEV light and the concomitant preventive or curative treatment of cataract and macular degeneration. There is very limited risk of post-procedure complications such as dry eye disease or post operative pain, as the corneal stromal tissue is not affected, neither are the corneal nerves damaged. Moreover, the present method provides a wider therapeutic window than subtractive refractive laser surgery as the biomaterial is added to the corneal surface, and the method is not dependent on the thickness of the cornea itself. For example, higher corrections are possible for hyperopia and presbyopia, irregular corneas can be treated, and higher astigmatism corrections than that with just laser can be achieved. Additionally, the present method allows for restoring/recovering a partly or completely damaged cornea resulting from chronic or acute corneal diseases.

The invention thus provides methods which involve the use of a combination of: (i) a gelatin functionalized with one or more tyrosine-derived or phenol groups, and (ii) a visible and/or UV light photoinitiator system, as a cross-linkable liquid composition for use in the treatment of eye disorders, whereby the cross-linkable liquid is cross-linked after application onto a surface of the eye such as the anterior corneal surface or the corneal stromal surface.

Hence, in embodiments of the uses or methods as taught herein, the method comprises:
applying the crosslinkable liquid composition onto an anterior corneal surface of the eye comprising the eye disorder,
irradiating the crosslinkable liquid composition on the eye comprising the eye disorder with visible and/or UV light, thereby obtaining a crosslinked composition on the anterior corneal surface of the eye of the subject, and optionally
correcting the curvature of the crosslinked composition,
wherein the method comprises introducing the crosslinkable liquid composition into a mold which is positioned onto the anterior corneal surface of the eye.

In embodiments of the uses or methods as taught herein, the method comprises:
applying the crosslinkable liquid composition onto a corneal stromal surface of the eye comprising the eye disorder,
irradiating the crosslinkable liquid composition on the eye comprising the eye disorder with visible and/or UV light, thereby obtaining a crosslinked composition on the corneal stromal surface of the eye of the subject, and optionally
cutting a lenticular shape out of the crosslinked composition,
wherein the crosslinkable liquid composition is applied onto a corneal stromal surface by intrastromal injection or after making surgical access to the corneal stromal surface.

In embodiments of the uses or methods as taught herein, the eye disorder is cataract, macular degeneration, a corneal refractive disorder, or a chronic or subacute corneal disease involving an irregularity of the cornea.

In embodiments of the uses or methods as taught herein, the corneal refractive disorder is selected from the group consisting of myopia, hyperopia, astigmatism, and presbyopia.

In embodiments of the uses or methods as taught herein, the chronic or subacute corneal disease involving an irregularity of the cornea is selected from the group consisting of corneal ulcer, corneal erosion, a corneal ectasia, and a corneal irregularity caused by trauma, surgery (e.g. corneal transplantation) or epithelial basement membrane disorder; preferably wherein the corneal ectasia is keratoconus.

In embodiments of the uses or methods as taught herein, the visible and/or UV light photoinitiator system comprises a photoinitiator and a co-initiator, such as wherein the photoinitiator comprises tris(2,2′-bipyridine)ruthenium(II) or riboflavin, and the co-initiator is sodium persulphate, such as wherein the photoinitiator is tris(2,2′-bipyridyl)dichlororuthenium(II) hexahydrate or riboflavin, and the co-initiator is sodium persulphate.

In embodiments of the uses or methods as taught herein, the gelatin functionalized with one or more tyrosine-derived or phenol groups is a gelatin desaminotyrosine.

In embodiments of the uses or methods as taught herein, the mold is a corneal vacuum suction device, a corneal bath or a contact lens.

In embodiments of the uses or methods as taught herein, the method comprises applying the mold onto the anterior corneal surface of the eye either before or after applying said crosslinkable liquid composition into the mold.

In embodiments of the uses or methods as taught herein, optionally, prior to applying the crosslinkable liquid composition onto the anterior corneal surface of the eye is (or prior to applying the mold), the anterior corneal surface of the eye is debrided, i.e., the epithelial cells are removed from the relevant part of the cornea.

In embodiments of the uses or methods as taught herein which involve the use of the mold, the method further comprises removing the mold after crosslinking the crosslinkable composition.

In embodiments of the uses or methods as taught herein, the crosslinkable liquid composition is applied as a single layer.

In embodiments of the uses or methods as taught herein, the crosslinked composition is a corneal onlay.

In embodiments of the uses or methods as taught herein, the crosslinked composition is a corneal inlay.

In embodiments of the uses or methods as taught herein, the crosslinked composition, such as the corneal onlay or corneal inlay, has a diameter of from about 6.0 mm to about 9.0 mm and a thickness of from about 10.0 μm to about 400.0 μm prior to correcting the curvature or shape of the crosslinked composition; and/or wherein the crosslinked composition, such as the corneal onlay or corneal inlay, is resistant to biodegradation for a period of at least 6 months, preferably for a period of at least 12 months.

A further aspect of the invention relates to a crosslinkable liquid composition for use in a surgical method of preparing a corneal onlay on or a corneal inlay in an eye of a subject, wherein the crosslinkable liquid composition comprises:
a crosslinkable biomaterial, wherein the crosslinkable biomaterial comprises a gelatin functionalized with one or more tyrosine-derived or phenol groups; and
a visible and/or UV light photoinitiator system.

In embodiments of the uses or methods as taught herein, the method comprises:
applying the crosslinkable liquid composition onto an anterior corneal surface of the eye comprising the eye disorder,
irradiating the crosslinkable liquid composition on the eye comprising the eye disorder with visible and/or UV light, thereby obtaining a corneal onlay on the anterior corneal surface of the eye of the subject, and optionally
correcting the curvature of the corneal onlay,
wherein the method comprises introducing the crosslinkable liquid composition into a mold which is positioned onto the anterior corneal surface of the eye;
or wherein the method comprises:
applying the crosslinkable liquid composition onto a corneal stromal surface of the eye comprising the eye disorder,
irradiating the crosslinkable liquid composition on the eye comprising the eye disorder with visible and/or UV light, thereby obtaining a corneal inlay on the corneal stromal surface of the eye of the subject, and optionally
cutting a lenticular shape out of the corneal inlay,
wherein the crosslinkable liquid composition is applied onto a corneal stromal surface by intrastromal injection or after making surgical access to the corneal stromal surface.

The above and further aspects and preferred embodiments of the invention are described in the following sections and in the appended claims. The subject-matter of appended claims is hereby specifically incorporated in this specification.

### DESCRIPTION OF THE DRAWINGS

FIG. 1: Schematic representation illustrating the different steps of an exemplary method according to an embodiment of the invention for preparing a corneal onlay for the treatment of a refractive error such as hypermetropic cornea (farsightedness) using a mold.

FIG. 2: Schematic representation illustrating the different steps of an exemplary method according to an embodiment of the invention for preparing a corneal onlay for the treatment of a refractive error such as hypermetropic cornea (farsightedness) using an o-ring.

FIG. 3: Schematic representation of exemplary steps of a method according to an embodiment of the invention for preparing a corneal inlay and adapting its form by sculpting it by intrastromal lenticular laser surgery (e.g. SMILE surgery).

FIG. 4: Schematic representation to compare methods of refractive correction according to traditional methods and according to an embodiment of the invention.

FIG. 5: Photographs illustrating corneal onlays with UV and HEV light blocking properties for use according to embodiments of the invention (A) before and (B) after washing.

FIG. 6: Graphs illustrating the transparency spectrum of: (A) GelDAT corneal onlays crosslinked with a visible and/or UV light photoinitiator system for use according to embodiments of the invention and (B) comparative GelMA corneal onlays with a UV light photoinitiator.

FIG. 7: Graph illustrating the crosslinking kinetics of two GelDAT crosslinkable compositions crosslinked with a visible and/or UV light photoinitiator system for use according to embodiments of the invention (dotted lines) and two comparative GelMA crosslinkable compositions crosslinked with a UV based photoinitiator (full lines). Black full line: 20% GelMA 90 kDa; grey full line: 20% GelMA 160 kDa; black dotted line: 20% GelDAT 90 kDa; grey dotted line: 15% GelDAT 160 kDa. Grey zone indicates the irradiation with a full spectrum light source OmniCure starting from 60 seconds until 350 seconds.

FIG. 8: Photographs illustrating corneal onlays with UV and HEV light blocking properties for use according to embodiments of the invention. The crosslinkable liquid compositions as taught herein comprise: (A) 15% (w/v) GelDAT+0.5 mM riboflavin/5 mM SPS, (B) 15% (w/v) GelDAT+1 mM riboflavin/5 mM SPS, and (C) GelDAT (w/v)+2 mM riboflavin/20 mM SPS.

FIG. 9: Graphs illustrating the transparency spectrum of: (A) GelDAT 120 kDa corneal onlays crosslinked with 0.4 mM (striped line), 0.8 mM (doted and striped line), 1.4 mM (dotted line) riboflavin for use according to embodiments of the invention. PBS (black full line) and blank (grey full line) were used as control. X-axis: wavelength (nm), Y-axis: transparency (%).

### DETAILED DESCRIPTION OF THE INVENTION

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms also encompass “consisting of” and “consisting essentially of”.

The recitation of numerical ranges by endpoints includes all numbers and fractions subsumed within the respective ranges, as well as the recited endpoints.

The term “about” as used herein when referring to a measurable value such as a parameter, an amount, a temporal duration, and the like, is meant to encompass variations of and from the specified value, in particular variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

Whereas the term “one or more”, such as one or more members of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members.

All documents cited in the present specification are hereby incorporated by reference in their entirety.

Unless otherwise specified, all terms used in disclosing the invention, including technical and scientific terms, have the meaning as commonly understood by one of ordinary skill in the art to which this invention belongs. By means of further guidance, term definitions may be included to better appreciate the teaching of the present invention.

The present invention is at least in part based on the inventors' innovative insight and experimental evaluation that a refractive error or a chronic or subacute corneal disease involving an irregularity of the cornea can be treated by applying a crosslinkable composition onto the anterior corneal surface of the affected eye as a liquid using a mold for holding the crosslinkable composition while applying it to the anterior corneal surface, subsequently crosslinking the composition in situ and, optionally, correcting the curvature of the newly formed corneal onlay, such as by photoablation, without subtracting the stroma of the cornea per se. Alternatively, a refractive error or a chronic or subacute corneal disease involving an irregularity of the cornea can be treated by applying a crosslinkable composition onto the corneal stromal surface of the affected eye as a liquid by intrastromal injection or after making surgical access to the corneal stromal surface, subsequently crosslinking the composition in situ and, optionally, correcting the shape of the newly formed corneal inlay, e.g., by cutting a lenticular shape out of the corneal inlay, such as by photoablation or Small-incision lenticule extraction (SMILE), without subtracting the stroma of the cornea per se. Accordingly, the method of present invention is less invasive than laser surgery performed directly on the cornea.

The methods of present invention provide an integrated, long-term, but reversible, solution for treating eye disorders, such as a refractive error or a chronic or subacute corneal disease involving an irregularity of the cornea, and has several advantageous over known methods for treating such eye disorders.

For example, unlike glasses or contact lenses, the method of present invention does not obstruct the subject during heavy duty work or contact sport, the crosslinked composition on the cornea obtained by the method present invention does not feel uncomfortable for the subject and, once applied, provides a long-term treatment of the eye disorder without risk of dry eyes and severe bacterial eye infections due to poor lens hygiene.

Furthermore, compared to subtractive refractive laser surgery performed directly on the eye, the method of the present invention has a very limited risk to post-procedure complications such as dry eye disease, post-operative pain or flap related problems, as the corneal stromal tissue is not affected nor is an epithelial flap is created. Moreover, the method of present invention provides a wider therapeutic window than subtractive refractive laser surgery performed on the cornea as biomaterial is added to the corneal surface and the method is not dependent on the thickness of the cornea itself. For example, higher corrections are possible for, for example, hyperopia and presbyopia, irregular corneas can be treated and high astigmatism correction can be achieved.

In embodiments, the method of present invention particularly aims at treating a subject's refractive error which was already present prior to treating the subject with the method as taught herein and not to treat a refractive error caused by the presence of the crosslinked material applied by the method as taught herein.

By experimental testing, the present inventors have found that the crosslinkable liquid composition comprising a gelatin functionalized with one or more tyrosine-derived or phenol groups and a visible and/or UV light photoinitiator system advantageously allows to obtain corneal onlays or corneal inlays with particularly advantageous properties such as UV and HEV light blocking properties and fast crosslinking kinetics.

The invention thus provides methods and compositions for use therein which involve the use of a combination of a gelatin functionalized with one or more tyrosine-derived or phenol groups and a visible and/or UV light photoinitiator system, such as a visible and/or UV light photoinitiator system comprising tris(2,2′-bipyridine)ruthenium(II), as a crosslinkable liquid composition for use in the treatment of eye disorders, whereby the cross-linkable liquid is crosslinked after application onto a surface of the eye.

Accordingly, the invention relates to a crosslinkable liquid composition comprising: (i) a gelatin functionalized with one or more tyrosine-derived or phenol groups and (ii) a visible and/or UV light photoinitiator system, for use in a method of curative or preventive treatment of an eye disorder in an eye of a subject.

Related aspects provide:
a method of curative or preventive treatment of an eye disorder in an eye of a subject in need of such a treatment, comprising administering a crosslinkable liquid composition comprising: (i) a gelatin functionalized with one or more tyrosine-derived or phenol groups and (ii) a visible and/or UV light photoinitiator system to the eye of a subject.
the use of a crosslinkable liquid composition comprising: (i) a gelatin functionalized with one or more tyrosine-derived or phenol groups and (ii) a visible and/or UV light photoinitiator system for the manufacture of a medicament for the curative or preventive treatment of an eye disorder in an eye of a subject.
the use of a crosslinkable liquid composition comprising: (i) a gelatin functionalized with one or more tyrosine-derived or phenol groups and (ii) a visible and/or UV light photoinitiator system for curative or preventive treatment of an eye disorder in an eye of a subject.

Preferably, the invention provides a crosslinkable liquid composition for use in a method of curative or preventive treatment of an eye disorder in an eye of a subject, wherein the method comprises:
applying the crosslinkable liquid composition onto an anterior corneal surface of the eye comprising the eye disorder,
irradiating the crosslinkable liquid composition on the eye comprising the eye disorder with visible and/or UV light, thereby obtaining a crosslinked composition on the anterior corneal surface of the eye of the subject, and optionally
correcting the curvature of the crosslinked composition,
wherein the crosslinkable liquid composition is introduced into a mold which is positioned onto the anterior corneal surface of the eye,
or wherein the method comprises:
applying the crosslinkable liquid composition onto a corneal stromal surface of the eye comprising the eye disorder,
irradiating the crosslinkable liquid composition on the eye comprising the eye disorder with visible and/or UV light, thereby obtaining a crosslinked composition on the corneal stromal surface of the eye of the subject, and optionally
cutting a lenticular shape out of the crosslinked composition,
wherein the crosslinkable liquid composition is applied onto a corneal stromal surface by intrastromal injection or after making surgical access to the corneal stromal surface,
wherein the crosslinkable liquid composition comprises: (i) a crosslinkable biomaterial, wherein the crosslinkable biomaterial comprises a gelatin functionalized with one or more tyrosine-derived or phenol groups; and (ii) a visible and/or UV light photoinitiator system.

The invention also relates to a method of treating an eye disorder in an eye of a subject, wherein the method comprises:
applying the crosslinkable liquid composition onto an anterior corneal surface of the eye comprising the eye disorder,
irradiating the crosslinkable liquid composition on the eye comprising the eye disorder with visible and/or UV light, thereby obtaining a crosslinked composition on the anterior corneal surface of the eye of the subject, and optionally
correcting the curvature of the crosslinked composition,
wherein the method comprises introducing the crosslinkable liquid composition into a mold which is positioned onto the anterior corneal surface of the eye;
or wherein the method comprises:
applying the crosslinkable liquid composition onto a corneal stromal surface of the eye comprising the eye disorder,
irradiating the crosslinkable liquid composition on the eye comprising the eye disorder with visible and/or UV light, thereby obtaining a crosslinked composition on the corneal stromal surface of the eye of the subject, and optionally
cutting a lenticular shape out of the crosslinked composition,
wherein the crosslinkable liquid composition is applied onto a corneal stromal surface by intrastromal injection or after making surgical access to the corneal stromal surface; and
wherein the crosslinkable liquid composition comprises: (i) a crosslinkable biomaterial, wherein the crosslinkable biomaterial comprises a gelatin functionalized with one or more tyrosine-derived or phenol groups; and (ii) a visible and/or UV light photoinitiator system.

The invention also relates to a crosslinkable liquid composition for use in a surgical method of preparing a corneal onlay on or a corneal inlay in an eye of a subject, wherein the crosslinkable liquid composition comprises:
a crosslinkable biomaterial, wherein the crosslinkable biomaterial comprises a gelatin functionalized with one or more tyrosine-derived or phenol groups; and
a visible and/or UV light photoinitiator system.

Related aspects provide:
a method of preparing a corneal onlay on or a corneal inlay in an eye of a subject in need of such a treatment, comprising administering a crosslinkable liquid composition comprising: (i) a crosslinkable biomaterial, wherein the crosslinkable biomaterial comprises a gelatin functionalized with one or more tyrosine-derived or phenol groups and (ii) a visible and/or UV light photoinitiator system to the eye of a subject.
the use of a crosslinkable liquid composition comprising: (i) a crosslinkable biomaterial, wherein the crosslinkable biomaterial comprises a gelatin functionalized with one or more tyrosine-derived or phenol groups and (ii) a visible and/or UV light photoinitiator system for the manufacture of a medicament for preparing of a corneal onlay on or a corneal inlay in an eye of a subject.
the use of a crosslinkable composition for preparing a corneal onlay or a corneal inlay on an eye of a subject, wherein the crosslinkable liquid composition comprises: (i) a crosslinkable biomaterial, wherein the crosslinkable biomaterial comprises a gelatin functionalized with one or more tyrosine-derived or phenol groups; and (ii) a visible and/or UV light photoinitiator system.

In embodiments of the uses or methods as taught herein, the methods as taught herein may comprise:
applying the crosslinkable liquid composition onto an anterior corneal surface of the eye comprising the eye disorder,
irradiating the crosslinkable liquid composition on the eye comprising the eye disorder with visible and/or UV light, thereby obtaining a corneal onlay on the anterior corneal surface of the eye of the subject, and optionally
correcting the curvature of the corneal onlay,
wherein the method comprises introducing the crosslinkable liquid composition into a mold which is positioned onto the anterior corneal surface of the eye.

In embodiments of the uses or methods as taught herein, the method comprises:
applying the crosslinkable liquid composition onto a corneal stromal surface of the eye comprising the eye disorder,
irradiating the crosslinkable liquid composition on the eye comprising the eye disorder with visible and/or UV light, thereby obtaining a corneal inlay on the corneal stromal surface of the eye of the subject, and optionally
cutting a lenticular shape out of the corneal inlay,
wherein the crosslinkable liquid composition is applied onto a corneal stromal surface by intrastromal injection or after making surgical access to the corneal stromal surface.

Accordingly, the invention also relates to a crosslinkable liquid composition for use in a surgical method of preparing a corneal onlay on or a corneal inlay in an eye of a subject, wherein the method comprises:
applying the crosslinkable liquid composition onto an anterior corneal surface of the eye comprising the eye disorder,
irradiating the crosslinkable liquid composition on the eye comprising the eye disorder with visible and/or UV light, thereby obtaining a corneal onlay on the anterior corneal surface of the eye of the subject, and optionally
correcting the curvature of the corneal onlay,
wherein the method comprises introducing the crosslinkable liquid composition into a mold which is positioned onto the anterior corneal surface of the eye;
or wherein the method comprises:
applying the crosslinkable liquid composition onto a corneal stromal surface of the eye comprising the eye disorder,
irradiating the crosslinkable liquid composition on the eye comprising the eye disorder with visible and/or UV light, thereby obtaining a corneal inlay on the corneal stromal surface of the eye of the subject, and optionally
cutting a lenticular shape out of the corneal inlay,
wherein the crosslinkable liquid composition is applied onto a corneal stromal surface by intrastromal injection or after making surgical access to the corneal stromal surface; and
wherein the crosslinkable liquid composition comprises: (i) a crosslinkable biomaterial, wherein the crosslinkable biomaterial comprises a gelatin functionalized with one or more tyrosine-derived or phenol groups; and (ii) a visible and/or UV light photoinitiator system.

A related aspect of the invention provides a method of preparing a corneal onlay or a corneal inlay on an eye of a subject, wherein the method comprises:
applying the crosslinkable liquid composition onto an anterior corneal surface of the eye
irradiating the crosslinkable liquid composition on the eye comprising the eye disorder with visible and/or UV light, thereby obtaining a corneal onlay on the anterior corneal surface of the eye of the subject, and optionally
correcting the curvature of the corneal onlay,
wherein the method comprises introducing the crosslinkable liquid composition into a mold which is positioned onto the anterior corneal surface of the eye;
or wherein the method comprises:
applying the crosslinkable liquid composition onto a corneal stromal surface of the eye comprising the eye disorder,
irradiating the crosslinkable liquid composition on the eye comprising the eye disorder with visible and/or UV light, thereby obtaining a corneal inlay on the corneal stromal surface of the eye of the subject, and optionally
cutting a lenticular shape out of the corneal inlay,
wherein the crosslinkable liquid composition is applied onto a corneal stromal surface by intrastromal injection or after making surgical access to the corneal stromal surface; and
wherein the crosslinkable liquid composition comprises: (i) a crosslinkable biomaterial, wherein the crosslinkable biomaterial comprises a gelatin functionalized with one or more tyrosine-derived or phenol groups; and (ii) a visible and/or UV light photoinitiator system.

Reference to “therapy” or “treatment” encompasses curative treatments, and the terms may particularly refer to the alleviation or measurable lessening of one or more symptoms or measurable markers of a pathological or abnormal condition such as a disease or disorder or a dysfunction (e.g. as result of surgery). Measurable lessening includes any statistically significant decline in a measurable marker or symptom. Generally, the terms encompass both curative treatments and treatments directed to reduce symptoms and/or slow progression of the disease. The term eye disorder thus generally encompasses both conditions caused by disease and conditions caused by other factors such as trauma or operation, which can affect the normal functioning of the eye.

The terms “subject”, “individual” or “patient” are used interchangeably throughout this specification, and typically and preferably denote humans, but may also encompass reference to non-human animals, preferably warm-blooded animals, even more preferably non-human mammals. Particularly preferred are human subjects including both genders and all age categories thereof. In other embodiments, the subject is an experimental animal or animal substitute as a disease model. The term does not denote a particular age or sex.

The term “subject in need of treatment” or similar as used herein refers to subjects diagnosed with or having a disease or disorder as recited herein.

In embodiments of the uses or methods as taught herein, the eye disorder may be cataract, macular degeneration, a corneal refractive disorder, or a chronic or subacute corneal disease involving an irregularity of the cornea.

In embodiments of the uses or methods as taught herein, the eye disorder may be cataract or macular degeneration.

The term “cataract” refers to a clouding or cloudy area in the lens of the eye that leads to a decrease in vision. Symptoms of cataract may include faded colours, blurry or double vision, halos around light, trouble with bright lights, and difficulty seeing at night.

The terms “macular degeneration”, “age-related macular degeneration”, “AMD”, or “ARMD” refer to a condition which may result in blurred or no vision in the center of the visual field. While early on there are often no symptoms, over time some people experience a gradual worsening of vision that may affect one or both eyes.

In embodiments of the uses or methods as taught herein, the eye disorder may be a corneal refractive disorder or a chronic or subacute corneal disease involving an irregularity of the cornea. In embodiments, the eye disorder may be a corneal refractive error. In embodiments of the uses or methods as taught herein, the corneal refractive disorder may be selected from the group consisting of myopia, hyperopia, astigmatism (e.g. regular or irregular astigmatism, corneal surgery-induced astigmatism or corneal transplantation-induced astigmatism), and presbyopia.

The term “corneal refractive error” or “corneal refraction error” as used herein refers to an eye disorder wherein the shape of the cornea prevents light from focussing correctly on the retinal fovea leading to impaired vision. Non-limiting examples of corneal refractive errors are nearsightedness (myopia), farsightedness (hyperopia), regular and irregular astigmatism, corneal surgery-induced astigmatism, corneal transplantation-induced astigmatism, and presbyopia. Symptoms caused by refractive errors include, but are not limited to, double vision, hazy vision, seeing a glare or halo around bright lights, squinting, headaches and eye strains. A refractive error may be diagnosed by any means known in the art, such as by use of an automated refractor. In preferred embodiments, the refractive error is of primary cause (e.g. naturally occurring or non-surgically induced) or of secondary cause which may include, but not limited to surgically induced (e.g. post-LASIK ectasia, post corneal transplantation astigmatism such as full thickness penetrating keratoplasty) as a result from infection, genetic factors, degenerative eye diseases or trauma to the eye.

The term “myopia” or “nearsightedness” as used herein has its meaning as generally accepted in the art. In myopia or nearsightedness, light rays are brought to focus in front of the retina. This may occur because the focusing power of the cornea and lens is very high and/or because the eyeball is too long from front to back. For myopia, subtractive photo-ablative laser surgery is safe and effective up to −10 diopters (dpt) in practice, but becomes less predictable in higher degrees of myopia and display higher complication rates. Accordingly, in particular embodiments, the refractive error is myopia with a diopter of more than −10.0, such as from −10.5 to −20.0. The corneal astigmatism may be corneal surgery-induced astigmatism or corneal transplantation-induced astigmatism.

The term “hyperopia” or “farsightedness” as used herein has its meaning as generally accepted in the art. In hyperopia or farsightedness, light rays are brought to focus behind the retina. This may occur because the focusing power of the cornea and lens is very low and/or because the eyeball is short in length from front to back. For hyperopia, subtractive photo-ablative laser surgery is safe and effective up to 3 diopters (dpt) in practice, but becomes less predictable in higher diopters. Accordingly, in particular embodiments, the refractive error is hyperopia with a diopter of more than +3.0, such as from +3.5 to +10.0, from +3.5 to +8.0, preferably from +3.5 to +4.0, +3.5, or +4.0.

The term “presbyopia” or “age-related farsightedness” as used herein has its meaning as generally accepted in the art. Presbyopia is physiological insufficiency of accommodation associated with the aging of the eye that results in progressively worsening ability to focus clearly on close objects. Presbyopia typically occurs due to age related changes in lens (decreased elasticity and increased hardness) and ciliary muscle of the eye, causing the eye to focus light behind rather than on the retina, when looking at close objects.

The term “astigmatism” as used herein has its meaning as generally accepted in the art, and includes regular, irregular astigmatism, simple astigmatism, compound astigmatism, myopic astigmatism, hyperopic astigmatism, mixed astigmatism, lenticular astigmatism, and corneal astigmatism. Astigmatism is a refractive error in the eye due to rotational asymmetry in the eye's refractive power. The underlying mechanism involves an irregular curvature of the cornea or abnormalities in the lens of the eye. In eyes without astigmatism, the cornea and lens have a more or less similar curvature in all directions. This allows light to be focused to a single point on the retina. People with astigmatism have more curvature in one direction, or meridian, than in another, so that light is not able to focus on a single point on the retina. This results in blurring of vision at all distances. For astigmatism, subtractive photo-ablative laser surgery is safe and effective up to 3 diopters (dpt) in practice, but becomes less predictable in higher diopters. Accordingly, in particular embodiments, the refractive error is astigmatism with a diopter of more than +3.0, such as from +3.5 to +16.0, from +3.5 to +8.0, preferably from +3.5 to +5.0, like +3.5, +4.0, +4.5, or +5.0.

There are different forms of corneal astigmatism. The corneal astigmatism may be corneal surgery-induced astigmatism or corneal transplantation-induced astigmatism.

Preferably, the astigmatism such as corneal astigmatism is a non-surgery induced or non-surgery related astigmatism. In embodiments, the astigmatism such as corneal astigmatism is not a surgical astigmatism caused by sutures, e.g., leading to refractive complications. In preferred embodiments, the astigmatism such as corneal astigmatism has a congenital origin.

In embodiments, the astigmatism may not be caused by an invasive treatment for correcting an eye disorder. Accordingly, in embodiments, the astigmatism may not be caused by refractive laser surgery, such as PRK, LASIK or SMILE.

In embodiments of the uses or methods as taught herein, the eye disorder may be cataract, macular degeneration, a corneal refractive error, or a combination thereof. In embodiments, the eye disorder may be cataract, macular degeneration, myopia, hyperopia, astigmatism, presbyopia, or a combination thereof. The uses and methods as taught herein advantageously allow for the treatment of a refractive error such as myopia, hyperopia, astigmatism, or presbyopia, while at the same time providing protection of the eyes from incoming UV and HEV light, thereby preventing or treating cataract and/or macular degeneration.

In embodiments, the eye disorder is a chronic or subacute corneal disease involving an irregularity of the cornea. Non-limiting examples of chronic or subacute corneal disease involving an irregularity of the cornea comprise corneal ulcers (e.g. caused by trauma or inflammation), corneal erosion, corneal ectactic disorders (i.e. corneal thinning) such as keratoconus, keratoglobus or post-LASIK ectasia, or a corneal irregularity inducing an irregular astigmatism such as a result from trauma or epithelial basement membrane dystrophy.

In embodiments of the uses or methods as taught herein, the chronic or subacute corneal disease involving an irregularity of the cornea may be selected from the group consisting of corneal ulcer, corneal erosion, a corneal ectasia, and a corneal irregularity caused by trauma or epithelial basement membrane disorder. Preferably, the corneal ectasia is keratoconus.

The terms “corneal ectasia” or “corneal ectatic disorder” as used herein refers to a group of uncommon, noninflammatory eye disorders characterised by bilateral thinning of the central, paracentral, or peripheral cornea.

In embodiments, the corneal ectasia may be selected form the group consisting of keratoconus, keratoglobus, pellucid marginal degeneration, posterior keratoconus, post-LASIK ectasia, and Terrien's marginal degeneration.

The term “keratoconus” refers to a progressive, noninflammatory, bilateral, asymmetric disease, characterized by paraxial stromal thinning and weakening that leads to corneal surface distortion.

In particular embodiments the chronic or subacute corneal diseases are not caused by an invasive treatment for correcting an eye disorder and in further particular embodiments, the patient has not previously undergone an invasive treatment for correcting an eye disorder in the eye that is to be treated according to the invention. Accordingly, in embodiments, the chronic or subacute corneal disease involving an irregularity of the cornea is not caused by or further to refractive laser surgery, such as PRK, LASIK or SMILE. In other embodiments of the uses or methods as taught herein, the chronic or subacute corneal disease involving an irregularity of the cornea may be caused by or be further to an invasive treatment for correcting an eye disorder. Hence, in embodiments, the chronic or subacute corneal disease involving an irregularity of the cornea may be caused by or may be subsequent to refractive laser surgery, such as PRK, LASIK or SMILE.

Likewise, in embodiments, the subject did not receive any invasive treatment for correcting the eye disorder in the eye, such as a refractive error, prior to the application of the crosslinkable liquid composition. In more particular embodiments, the subject did not receive refractive laser surgery, such as PRK, LASIK or SMILE, prior to the application of the crosslinkable liquid composition onto the corneal surface of the eye.

Of particular interest are subjects whose eyes are frequently exposed to UV and/or HEV light (e.g., at least 4 hours per day, such as at least 6 hours or at least 8 hours per day), those subjects of which the eye disorder cannot be treated using existing techniques, such as subjects that are excluded from treatment by refractive laser therapy, such as subjects with a very thin cornea, subjects with persistent dry eyes or subjects performing contact sports. Accordingly, in embodiments, the subject has a cornea thickness of less than 480.0 μm, less than 450.0 μm or less than 400.0 μm.

Crosslinking is the formation of chemical links between molecular chains to form a three-dimensional network of connected molecules. Crosslinks may be formed by chemical reactions that are initiated in the presence of the visible and/or UV light photoinitiator system comprising a photoinitiator as described herein, which typically comprises multiple functional groups and forms radicals upon irradiation, thereby as such starting the crosslinking reaction.

The “crosslinkable liquid” or “crosslinkable liquid composition” as referred to herein comprises crosslinkable biocompatible material (e.g. biomaterial). The terms “liquid” or “liquid composition” in the context of the present invention encompass both completely liquid and semi-liquid compositions, i.e. include compositions which have a consistency between solid and liquid.

The crosslinkable liquid composition to be used in the method of present invention comprises:
a crosslinkable biomaterial, wherein the crosslinkable biomaterial comprises, consists essentially of, or consists of a gelatin functionalized with one or more tyrosine-derived or phenol groups; and
a visible and/or UV light photoinitiator system. Preferably, the crosslinkable biomaterial is a gelatin functionalized with one or more tyrosine-derived or phenol groups.

The crosslinkable liquid composition to be used in the method of present invention advantageously comprises a combination of a gelatin functionalized with one or more tyrosine-derived or phenol groups and a visible and/or UV light photoinitiator system that can crosslink (upon induction by visible and/or UV light) and can take up substantial amounts of water from its surroundings without dissolving at body temperature, such at about 37° C.

In embodiments, the crosslinkable biomaterial comprises, consists essentially of, or consists of a gelatin functionalized with one or more tyrosine-derived or phenol groups. Preferably, the crosslinkable biomaterial is a gelatin functionalized with one or more tyrosine-derived or phenol groups.

The term “gelatin” refers to a composition comprising or consisting of proteins and obtained by partial hydrolysis of collagen extracted from the skin, bones, and connective tissues of animals.

In embodiments, the gelatin functionalized with one or more tyrosine-derived or phenol groups may have a degree of substitution of from about 40% to about 90%. For instance, the gelatin functionalized with one or more tyrosine-derived or phenol groups may have a degree of substitution of from about 50% to about 80%, or from about 60% to about 70%.

The “degree of substitution” of a functionalized gelatin refers to the ratio (expressed as a percentage) of the number of modified amino groups to the number of free amino groups of the gelatin. The phrases “degree of substitution”, “degree of modification” or “degree of functionalization” may be used interchangeably herein.

In embodiments, the one or more tyrosine-derived or phenol groups may be desaminotyrosine, desaminotyrosyl tyrosine, or tyramine.

The terms “desaminotyrosine”, “DAT”, “phloretic acid”, “phloretate” or “hydro-p-coumaric acid” can be used interchangeably herein. Desaminotyrosine has 3-(4-hydroxyphenyl)propanoic acid as IUPAC name.

The terms “desaminotyrosyl tyrosine”, “desaminotyrosyl-tyrosine” or “DATT” may be used interchangeably. The IUPAC name is (2R,4S)-4-amino-5-(4-hydroxyphenyl)-2-[(4-hydroxyphenyl)methyl]-3-oxopentanoic acid.

The terms “tyramine” or “4-hydroxyphenethylamine” can be used interchangeably herein. Tyramine has 4-(2-aminoethyl)phenol as the IUPAC name.

In embodiments of the uses or methods as taught herein, the gelatin functionalized with one or more tyrosine-derived or phenol groups may be selected from the group consisting of a gelatin desaminotyrosine, a gelatin desaminotyrosyl tyrosine, or a gelatin tyramine. Preferably, the gelatin functionalized with one or more tyrosine-derived or phenol groups is a gelatin desaminotyrosine. Such gelatins advantageously allow crosslinking with the visible and/or UV light photoinitiator with very fast crosslinking kinetics and provide a crosslinked composition such as an onlay or inlay with UV and HEV light blocking properties.

The terms “gelatin desaminotyrosine” (bearing one aromatic ring per free amino group of gelatin), “gelatin functionalized with desaminotyrosine” or “GelDAT” may be used interchangeably herein.

Suitable gelatin desaminotyrosine includes X-Pure GelDAT commercially available from Rousselot BV, Ghent, Belgium.

The term “gelatin desaminotyrosyl tyrosine” (bearing two aromatic rings per free amino group of gelatin), “gelatin functionalized with desaminotyrosyl tyrosine” or “GelDATT” may be used interchangeably herein.

Suitable gelatin desaminotyrosyl tyrosine can be synthesized as described in Roch et al. (2011, Macromol. Symp., 309, 182-189), in particular on p. 184, Materials and Methods, Functionalization of gelatin. For instance, desaminotyrosine or desaminotyrosyl tyrosine (29 mmol) may be activated by reaction with 1-ethyl-3-(3-dimethyl-aminopropyl) carbodiimide (EDC) (32 mmol) and N-hydroxysuccinimide (NHS) (43 mmol) in 110 mL of dimethyl sulfoxide (DMSO) at 37° C. After 3 h, β-mercaptoethanol (43 mmol) may be added. A gelatin solution (15 g in 150 mL DMSO) may be added and the mixture stirred at 37° C. for 5 h. The functionalized product may be precipitated in ethanol, filtered, washed with ethanol and acetone, and dried under vacuum.

The terms “gelatin tyramine”, “gelatin functionalized with tyramine” or “GTA” may be used interchangeably herein.

Suitable gelatin tyramine can be synthesized as described in Sakai et al. (2009, Biomaterials, 30, 3371-3377), in particular on p. 3372, Materials and methods, 2.2 Modification of gelatin to incorporate phenol groups, or as described in Li et al. (2015, Acta Biomater., 13, 88-100), in particular in 2. Materials and methods, 2.2 Synthesis of gelatin/tyramine/heparin (G/T/H) conjugates. For instance, gelatin derivatives possessing phenol (Ph) groups may be synthesized by combining gelatin and tyramine hydrochloride via the carbodiimide-mediated condensation of the carboxyl groups of gelatin and the amino groups of tyramine. Gelatin powder may be suspended at 2% (w/v) in a 50 mM morpholinoethanesulfonic acid (MES) aqueous solution and heated to 60° C. After dissolution of gelatin, the solution may be cooled to 25° C. To this solution, tyramine hydrochloride, EDC and NHS may be added and the solution may be stirred at 25° C. After 12 h of stirring, 50 mM sodium phosphate may be added. After a further 30 min of stirring, the resultant polymer solution may be dialyzed against deionized water, using an ultrafiltration membrane (MWCO: 10,000), until an absorbance peak at 275 nm, attributed to the presence of residual tyramine, is undetectable in the filtered solution. The sample may subsequently be lyophilized.

In embodiments of the uses or methods as taught herein, the crosslinkable liquid composition may comprise at least about 1.0% (w/v) of the gelatin functionalized with one or more tyrosine-derived or phenol groups. In embodiments, the crosslinkable liquid composition may comprise at least about 5.0% (w/v), at least about 10.0% (w/v), at least about 15.0% (w/v), or at least about 20.0% (w/v) of the gelatin functionalized with one or more tyrosine-derived or phenol groups. Such concentrations of the functionalized gelatin provide satisfactory crosslinking kinetics and good UV and HEV light blocking properties.

The terms “% w/v”, “% m/v”, “percentage weight per volume” or “percentage mass per volume” may be used interchangeably and refer to the ratio of the mass of a solid (the solute) (in grams) to the volume of the solution (in ml) times 100. For instance, the crosslinkable liquid composition may comprise about 10 g of GelDAT per 100 ml of solution. If 10 g of GelDAT is used to make up a total volume of 100 ml, then a 10% w/v solution of GelDAT has been made.

In embodiments, the crosslinkable liquid composition may comprise from about 1.0% to about 40.0% (w/v), from about 1.0% to about 30.0% (w/v), from about 1.0% to about 20.0% (w/v), from about 1.0% to about 15.0% (w/v), or from about 1.0% to about 10.0% (w/v) of the gelatin functionalized with one or more tyrosine-derived or phenol groups. In embodiments of the uses or methods as taught herein, the crosslinkable liquid composition may comprise from about 5.0% to about 40.0% (w/v) of the gelatin functionalized with one or more tyrosine-derived or phenol groups. In embodiments, the crosslinkable liquid composition may comprise from about 5.0% to about 30.0% (w/v), from about 5.0% to about 20.0% (w/v), from about 5.0% to about 15.0% (w/v), or from about 10.0% to about 20.0% (w/v) of the gelatin functionalized with one or more tyrosine-derived or phenol groups. Such concentrations of the functionalized gelatin provide satisfactory crosslinking kinetics and good UV and HEV light blocking properties.

In embodiments of the uses or methods as taught herein, the gelatin functionalized with one or more tyrosine-derived or phenol groups has a molecular mass of about 50000 g/mol (50 kDa) to about 200000 g/mol (200 kDa). For instance, the gelatin functionalized with one or more tyrosine-derived or phenol groups has a molecular mass of about 60 kDa to about 190 kDa, about 70 kDa to about 180 kDa, or about 80 kDa to about 170 kDa. Preferably, the gelatin functionalized with one or more tyrosine-derived or phenol groups has a molecular mass of about 90 kDa to about 160 kDa. In further embodiments the gelatin functionalized with one or more tyrosine-derived or phenol groups has a molecular mass of about 90 kDa and/or about 160 kDa. Such molecular masses of the functionalized gelatin as taught herein allow satisfactory crosslinking kinetics and good UV and HEV light blocking properties.

In embodiments, the crosslinkable liquid composition as taught herein may further comprise an aqueous solution, in particular a buffer solution, such as phosphate buffered saline (PBS).

The term “aqueous solution” refers to any solution comprising water or in which the solvent is water. Additionally, “aqueous solution” is used to describe solutions displaying commonalities to water or watery solutions, not limited to characteristics such as appearance, smell, colour, taste, viscosity, pH, absorbance, or physical state under particular temperatures.

The term “buffer component”, “buffer solution”, or “buffer” as used interchangeably herein refers to an aqueous solution comprising a mixture of a weak acid and its conjugate base or vice versa. Buffer solutions are characterized by their means to keeping the pH of a solution nearly constant when limited amounts of strong acids or strong bases are added to the solution. The amount of strong acid or strong base that can be added to the buffer solution before a significant pH change occurs is dependent on the specific buffer solution used and is commonly referred to as the buffer capacity. The pH of a buffer solution can be estimated using the Henderson-Hasselbalch equation, which is known to a person skilled in the art.

In certain embodiments, the crosslinkable liquid composition as taught herein may comprise from about 10% to about 98% by weight of an aqueous solution, in particular a buffer solution such as PBS. In embodiments, the crosslinkable liquid composition as taught herein may comprise from about 20% to about 98%, from about 30% to about 98%, from about 40% to about 98%, from about 50% to about 98%, from about 60% to about 98%, from about 60% to about 95% or from about 60% to about 90% by weight of an aqueous solution, in particular a buffer solution such as PBS.

The terms “weight percentage”, “mass percentage”, “percentage (%) by weight”, “weight %” or “wt %” indicate the mass of a substance to the total mass of the formulation (i.e. mass fraction) with a denominator of 100. Unless indicated otherwise, the wt % is provided herein compared to the total weight of the crosslinkable liquid composition.

In embodiments, the crosslinkable liquid composition as taught herein may not comprise organic solvents. In embodiments, the crosslinkable liquid composition as taught herein may be prepared in an aqueous carrier without the use of organic solvents.

The crosslinkable liquid composition may further also comprise one or more other crosslinking agents, such as a photoinitiator, that participate in the crosslinking reaction.

The crosslinking liquid composition further comprises a photoinitiator system. Hence, the crosslinking herein is advantageously performed by photocrosslinking.

Photoinitiators are compounds that upon radiation of light decompose into reactive species that activate polymerization of specific functional groups on the crosslinkable biomaterial. Accordingly, photoinitiators are typically used when the crosslinkable liquid composition is capable of being crosslinked by photocrosslinking. The type of visible and/or UV light photoinitiator as well as the concentration thereof can be varied in the crosslinkable liquid composition as intended herein. Each specific visible and/or UV light photoinitiator is typically linked to an excitation wavelength spectrum, of which the peak of the spectrum is the most optimal wavelength to create radicals upon excitation.

In embodiments of the uses or methods as taught herein, the visible and/or UV light photoinitiator system comprises a photoinitiator, in particular a visible and/or UV light photoinitiator. In embodiments, the visible and/or UV light photoinitiator may be selected from the group consisting of a tris(2,2′-bipyridine)ruthenium(II) complex, riboflavin, lithium phenyl-2,4,6-trimethylbenzoylphosphinate (lithium aryl phosphinate or LAP), and camphorquinone.

The visible and/or UV light photoinitiators suitable for use in the present invention are preferably UV/blue light photoinitiators in that they advantageously have an excitation spectrum in the UV/blue light spectrum, (380-500 nm) to create radicals that are used in the polymerization reaction, thereby leading to their UV/blue light absorption. The crosslinked compositions block UV/HEV light and appear to be yellow (FIG. 5). The wavelength of visible and/or UV light used in photochemical reactions is determined by the absorption spectrum of the photoinitiator. For instance, a tris(2,2′-bipyridine)ruthenium(II) complex has a maximal absorption wavelength of 452 nm. Riboflavin has a maximal absorption wavelength of 444 nm. LAP has a maximal absorption wavelength of 405 nm. Camphorquinone has a maximal absorption wavelength if 450 nm. The compositions of the present invention may thus be crosslinked by UV light and/or blue light, so as to generate enough radicals to start the cross-linking.

In embodiments of the uses or methods as taught herein, the visible light photoinitiator system comprises a photoinitiator and a co-initiator. In embodiments of the uses or methods as taught herein, the visible light photoinitiator system comprises one or more photoinitiators and one or more co-initiators. In particular embodiments, a photoinitiator is riboflavin or flavin. In particular embodiments, the photoinitiator is a tris (2,2′-bipyridyl) ruthenium (II) compound.

During photoactivation, the photolysis of Ru2+ generates excited Ru3+ that in turn oxidize aromatic residues, such as tyrosine. These oxidized tyrosine groups are further converted into tyrosyl radicals, then subsequently quenched by forming di-tyrosine bonds with other nearby tyrosine groups. This specific photoredox reaction is not only efficient, but also very rapid, mainly due to the high absorbance of Ru in the visible light range and high chemical stability in the excited state. In particular embodiments, the photoinitiator comprises tris(2,2′-bipyridine)ruthenium(II) or riboflavin, and the co-initiator is a sulfinate or sulfonate such as sodium persulphate. In embodiments, the visible or UV light photoinitiator system is a visible light photoinitiator system. In embodiments, the visible light photoinitiator system may comprise riboflavin as the photoinitiator and sodium persulphate as the co-initiator. In embodiments, the visible light photoinitiator system may comprise tris(2,2′-bipyridyl)dichlororuthenium(II) hexahydrate as the photoinitiator and sodium persulphate as the co-initiator. In embodiments, the visible or UV light photoinitiator system comprises tris(2,2′-bipyridine)ruthenium(II) or riboflavin. In embodiments, the visible or UV light photoinitiator system comprises tris(2,2′-bipyridine)ruthenium(II) or riboflavin, and sodium persulphate. In embodiments, the visible or UV light photoinitiator system comprises tris(2,2′-bipyridine)ruthenium(II). In embodiments, the visible or UV light photoinitiator system comprises tris(2,2′-bipyridine)ruthenium(II) and sodium persulphate.

In embodiments, the visible or UV light photoinitiator system comprises a photoinitiator and a co-initiator, wherein the photoinitiator comprises tris(2,2′-bipyridine)ruthenium(II) and the co-initiator is sodium persulphate. In embodiments, the visible or UV light photoinitiator system comprises a photoinitiator and a co-initiator, wherein the photoinitiator is tris(2,2′-bipyridyl)dichlororuthenium(II) hexahydrate and the co-initiator is sodium persulphate.

The terms “tris(2,2′-bipyridyl)dichlororuthenium(II) hexahydrate”, “tris(2,2-bipyridyl)dichlororuthenium(II) hexahydrate”, “Ru(BPY)3”, “ruthenium-tris(2,2′-bipyridyl) dichloride”, “tris(2,2′-bipyridyl)ruthenium(II) chloride hexahydrate” may be used interchangeably herein. The CAS number is 50525-27-4. The IUPAC name is 2-pyridin-2-ylpyridine; ruthenium(2+); dichloride; hexahydrate (as computed by Lexichem TK 2.7.0, PubChem release 2021.05.07).

Flavins, such as riboflavin and flavin are naturally occurring yellow pigments which are photoreducible, have high water solubility and are biocompatible. Riboflavin has an absorption peak of 440 nm and 371 nm.

Hence, an aspect provides a crosslinkable liquid composition for use in a method of curative or preventive treatment of an eye disorder in an eye of a subject, wherein the crosslinkable liquid composition comprises:
a crosslinkable biomaterial, wherein the crosslinkable biomaterial comprises a gelatin functionalized with one or more tyrosine-derived or phenol groups; and
tris(2,2′-bipyridine)ruthenium(II) or riboflavin, and optionally a co-initiator such as sodium persulphate.

A further aspect provides a crosslinkable liquid composition for use in a method of curative or preventive treatment of an eye disorder in an eye of a subject, wherein the crosslinkable liquid composition comprises:
a crosslinkable biomaterial, wherein the crosslinkable biomaterial comprises a gelatin functionalized with one or more tyrosine-derived or phenol groups; and tris(2,2′-bipyridine)ruthenium(II), and optionally a co-initiator such as sodium persulphate.

In embodiments, the crosslinkable composition comprises from 0.1 to 2.0% (w/v) such as from 0.05 to 2.0% (w/v), more particularly from 0.5 to 20% or from 1.0 to 2.0% (w/v) of the visible light photoinitiator.

Suitable co-initiators for the photo-initiators described herein are known in the art. Examples include a sulfinate or sulfonate such as sodium persulphate, amines such as L-arginine.

In particular embodiments a concentration of between 0.5 and 2 mM of photoinitiator is used with a concentration of 5-20 mM co-initiator, such as 1 mM photoinitiator with 10 mM co-initiator.

In further particular embodiments a concentration of between 0.5 and 1 mM of tris(2,2′-bipyridine)ruthenium(II) photoinitiator is used with a concentration of 5-10 mM sodium persulphate, such as 1 mM tris(2,2 ′-bipyridine)ruthenium(II) photoinitiator with 10 mM sodium persulphate. In further particular embodiments a concentration of between 0.5 and 2 mM of riboflavin photoinitiator is used with a concentration of 5-20 mM sodium persulphate, such as 1 mM riboflavin photoinitiator with 10 mM sodium persulphate. In embodiments of the uses or methods as taught herein, the crosslinkable liquid composition comprises a gelatin desaminotyrosine, a photoinitiator, and optionally a co-initiator; such as wherein the photoinitiator comprises tris(2,2′-bipyridine)ruthenium(II) or riboflavin, and the co-initiator is sodium persulphate. In embodiments of the uses or methods as taught herein, the crosslinkable liquid composition comprises a gelatin desaminotyrosine, tris(2,2′-bipyridyl)dichlororuthenium(II) hexahydrate, and sodium persulphate. In embodiments, the crosslinkable liquid composition comprises a gelatin desaminotyrosine, riboflavin, and sodium persulphate.

In embodiments, the crosslinkable liquid composition may further comprise one or more therapeutic agents (e.g. an analgesic, an anti-inflammatory agent, an antibiotic, a growth factor to stimulate epithelialization, or a steroid), and/or other agents such as colorants.

Once the crosslinkable liquid composition has been crosslinked, the crosslinked composition preferably does not interfere with the normal functionality of the eye and provides sufficient nutrient and gas exchange to maintain a viable corneal epithelium and stroma. Accordingly, in embodiments, the crosslinked composition is permeable to water, nutrients, oxygen, therapeutic agents (e.g. an analgesic, an anti-inflammatory agent, an antibiotic, a growth factor to stimulate epithelialization, or a steroid), and/or growth factors (e.g. exogenous or endogenous growth factors, such as nerve growth factor (NGF)).

Preferably, the crosslinked composition is compatible with clinical imaging techniques, such as clinical corneal investigation using a refractometer, optical coherence tomography, Scheimpflug tomography, Placido based tomography device or in vivo confocal imaging.

The crosslinked composition as taught herein such as the corneal onlay or corneal inlay may advantageously block UV and/or HEV light radiation. In embodiments, the crosslinked composition may have a transparency of at most 40%, such as at most 30%, at most 20%, at most 10%, at most 5%, or no transparency at all, when measured using light with a wavelength spectrum of at most 500 nm, such as when measured using light with a wavelength spectrum of 380-500 nm. In embodiments, the crosslinked composition may have a transparency of at most 40%, such as at most 30%, at most 20%, at most 10%, at most 5%, or no transparency at all, when measured using light with a wavelength spectrum of at most 490 nm or at most 480 nm, such as when measured using light with a wavelength spectrum of 380-480 nm. In embodiments, the crosslinked composition may have a transparency of at most 40%, such as at most 30%, at most 20%, at most 10%, at most 5%, or no transparency at all, when measured using light with a wavelength spectrum of at most 470 nm, at most 460 nm, or at most 450 nm, such as when measured using light with a wavelength spectrum of 380-470 nm.

In embodiments, the crosslinked composition has a transparency of at least 40%, at least 50%, at least 60%, at least 70%, at least 80% or at least 90%, when measured using light with a wavelength spectrum of above 500 nm, e.g. of at least 550 nm. The transparency may be determined by any means in the art, such as by a microplate reader or spectrophotometer such as described by Rizwan et al. (Biomaterials, 2017, 120:139-154) or by Van Hoorick et al. (Adv. Healthcare Materials, 2020, 9(6): 2000760).

In embodiments, the crosslinkable liquid composition and/or crosslinked composition has a refractive index similar to that of the native corneal stroma. The refractive index may be measured by any means in the art, such as by use of a refractometer.

Upon crosslinking of the crosslinkable liquid composition, the crosslinked composition will hold water within its three-dimensional network of polymers, resulting in the formation of a hydrogel.

The term “hydrogel” as used herein has its meaning as known in the art and refers to a biphasic material, a mixture of porous, permeable solids and at least 10% by weight or volume of interstitial fluid composed completely or mainly by water. In hydrogels the porous permeable solid is a water insoluble three dimensional network of polymers and a fluid, having absorbed a large amount of water or biological fluids. The term “hydrogel” may be used interchangeably herein with the term “crosslinked composition”.

The “swelling ratio” may be defined as the fractional increase in the weight of the crosslinked composition due to water absorption. The swelling ratio may be influenced by the type of the crosslinkable biomaterial, the concentration of the crosslinkable biomaterial within the crosslinkable liquid composition, and the degree of functionalisation of the biomaterial.

In embodiments, the crosslinked composition may have a swelling ratio of 200% to 1000%, when fully hydrated. In embodiments, the crosslinked composition may have a swelling ratio of 300-800%, 400-800%, or 400-600%, when fully hydrated.

One of the main advantages of the uses or methods as taught herein over existing techniques is that it provides a long-term solution for treating a refractive error or a chronic or subacute corneal disease involving an irregularity of the cornea. Accordingly, in embodiments, the crosslinked composition may be stable at body temperature, such at about 37° C., preferably for a period of at least 1 month, at least 2 months, at least 3 months, at least 4 months, at least 5 months, at least 6 months, at least 1 year, at least 5 years, or at least 10 years.

Present inventors have found that by applying the crosslinkable liquid composition to the anterior corneal surface of the eye using a mold, the crosslinkable liquid composition and final crosslinked composition, can be precisely placed onto the anterior corneal surface of the eye while avoiding spilling of the crosslinkable liquid composition over the entire anterior eye surface and/or under the eyelids before crosslinking. Furthermore, the mold may already give a primary shape, diameter and/or preliminary curvature to the final corneal onlay that will be formed by the crosslinked composition. By creating the corneal onlay in situ, it perfectly fits the patient's corneal geometry, compared to a corneal onlay created separately.

In embodiments of the uses or methods as taught herein, the crosslinking is performed in situ, onto the anterior corneal surface of the eye. Hence, in embodiments of the uses or methods as taught herein, the crosslinked composition is a corneal onlay. In embodiments, the crosslinking is performed at body temperature, such as at a temperature of from 35.0° C. to 40.0° C. or from 36.0° C. to 38.0° C.

The term “corneal onlay” generally refers to an optical device positioned on the eye, particularly between the epithelium and the Bowman's membrane of the cornea.

In embodiments of the uses or methods as taught herein, when the crosslinked composition is a corneal onlay, the method may comprise:
applying the crosslinkable liquid composition onto an anterior corneal surface of the eye comprising the eye disorder,
irradiating the crosslinkable liquid composition on the eye comprising the eye disorder with visible light, thereby obtaining a crosslinked composition, in particular a corneal onlay, on the anterior corneal surface of the eye of the subject, and
correcting the curvature of the crosslinked composition, in particular the corneal onlay,
wherein the method comprises introducing the crosslinkable liquid composition into a mold which is positioned onto the anterior corneal surface of the eye.

In embodiments of the uses or methods as taught herein, the mold may be a corneal vacuum suction device, a corneal bath or a contact lens. In embodiments, the mold is a corneal vacuum suction device. In embodiments, the mold is an O-ring shaped corneal bath (also referred to as an o-ring). In alternative embodiments, the mold is a contact lens, preferably a contact lens, more preferably a scleral contact lens, that does not adhere to the biomaterial either in un-crosslinked or crosslinked state. In embodiments, the mold is a silicone hydrogel contact lens. A contact lens with a converging meniscus-shape allows shaping the crosslinked composition (i.e. biomaterial) into a lens-shape having a lens body (or lens (optical) zone) and a lens edge at the periphery of the lens body (or peripheral zone). Accordingly, the shaped crosslinked composition would use such a contact lens as a mold that is thinner towards its peripheral edges, and hence, typically having a peripherical edge which is less thick than when use is made of a corneal bath. As a result thereof, less or even no biomaterial will need to be removed upon correcting the curvature of the crosslinked composition, thereby making the uses or methods as taught herein more efficient and attractive.

In embodiments, the mold is a standard contact lens, which, the inventors have found, when used in the uses or methods as taught herein, inherently generates in an onlay for which the periphery is thicker than the center.

In embodiments, the shape of the mold may be adapted depending on the eye disorder to be treated. For example, for the treatment of farsightedness, the crosslinkable liquid composition can be added predominantly centrally of the cornea and for the treatment of near-sightedness, the crosslinkable liquid composition can be added predominantly peripherally of the cornea.

The shape of the mold may take into account the swelling of the crosslinkable liquid composition that may occur upon crosslinking and uptake of water by the biomaterial. For example, if a crosslinkable biomaterial is known to swell to twice its size upon crosslinking, and if a thickness of about 50.0 μm of the crosslinked composition (prior to eventual correcting the curvature) would be desired, the mold may be designed to only allow applying a layer of crosslinkable liquid composition with a thickness of about 25.0 μum.

In embodiments, the curvature of the contact lens may have a back central zone radius of from 8.0 to 15.0 mm, from 8.0 to 14.0 mm, from 8.0 to 13.0 mm, from 8.0 to 12.0 mm, from 8.0 to 11.0 mm, or from 8.0 to 10.0 mm.

Preferably, the curvature of the contact lens is so that it allows forming a void space or lens shaped cavity between the anterior surface of the cornea of the subject to be treated and the back surface of the central (optical) zone of the contact lens, while the back surface of the peripheral zone closely aligns with the peripheral zone of the cornea or sclera. This void space or lens shaped cavity can be filled with the crosslinkable liquid composition as described herein.

In embodiments, the shape of the mold is so that it allows the generation of a crosslinked composition or the subsequent shaping the crosslinked composition as to have an average thickness of from about 10.0 μm to about 400.0 μm, such as from about 20.0 μm to about 400.0 μm, from about 25.0 μm to about 400.0 μm, from about 50.0 μm to about 400.0 μm, from about 100.0 μm to about 400.0 μm, from about 200.0 μm to about 400.0 μm, or from about 100.0 μm to about 300.0 μm, prior to eventual correcting the curvature of the crosslinked composition.

In embodiments, the mold may cover at least 80%, at least 85%, at least 90%, or at least 95%, such as at least 95%; at least 96%, at least 97%, at least 98%, at least 99% or 100%, of the anterior corneal surface of the eye. In embodiments, the mold may completely cover the anterior corneal surface of the eye.

In embodiments, the shape of the mold may be such that it allows shaping the crosslinked composition as to have a diameter of from about 6.0 mm to about 9.0 mm, such as from about 6.0 mm to about 8.0 mm or from about 7.0 mm to about 9.0 mm. Present inventors realized that a wider diameter could risk covering the limbal epithelial cells, which differentiate and migrate to become corneal epithelial cells. Therefore, physically covering the limbus forms a risk of corneal epithelial cell ingrowth or impedes limbal stem cell differentiation, which is preferably avoided.

In embodiments, the mold, preferably the contact lens, has a total diameter (including the diameter of the central zone as well as peripheral zone of the mold) of from about 5.0 mm to about 30.0 mm, from 5.0 mm to 25.0 mm, from 10.0 mm to 25.0 mm, from 14.0 mm to 24.0 mm, from 5.0 mm to 10.0 mm, from 6.0 mm to 9.0 mm, or from 7.0 mm to 8.0 mm.

In embodiments, the mold is capable of allowing the UV and/or visible light to reach the crosslinkable liquid composition. For example, if the mold is a contact lens, the contact lens allows passage of visible light.

In embodiments of the uses or methods as taught herein, the method may comprise applying the mold onto the anterior corneal surface of the eye either before or after applying said crosslinkable liquid composition into the mold. In embodiments of the uses or methods as taught herein, the mold may be a corneal vacuum suction device, a corneal bath, or a contact lens and the method may comprise applying the mold onto the anterior corneal surface of the eye either before or after applying said crosslinkable liquid composition into the mold.

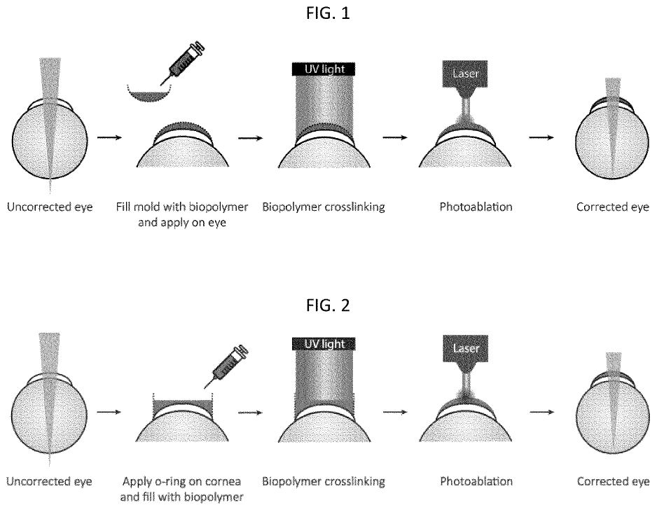

In embodiments, the mold such as a contact lens may be filled with the crosslinkable liquid composition prior to applying the mold and crosslinkable liquid composition to the anterior corneal surface of the eye, as illustrated in FIG. 1. In embodiments, the mold such as an O-ring shaped corneal bath (also referred to as an “o-ring”) may be applied to the anterior corneal surface of the eye prior to filling the mold with the crosslinkable liquid composition, as illustrated in FIG. 2.

In particular embodiments, a vacuum suction device is used. The vacuum suction device acts similarly as the o-ring, the difference is that the suction device is secured on the cornea and consists in different diameters to apply the onlay. In practice, the device is vacuum locked on top of the eye, the crosslinkable liquid composition is added and irradiated. Thereafter the suction device is removed. In more particular embodiments, the method comprises the steps of removing the corneal epithelium, pressing down the vacuum syringe (coupled to the vacuum ring) and placing the ring centrally on the cornea, gently releasing the vacuum syringe to attach the vacuum ring to the eye, placing the crosslinkable fluid composition inside the ring and crosslinking it with the light.

Alternatively the surgeon can opt to remove the epithelium after placing the ring (to only remove the epithelial cells in that area. A vacuum suction device is for instance illustrated in FIG. 1B of Kim et al. (J. Vet Sci, 2015, 16, 349-356). In embodiments, the crosslinkable liquid composition may be applied onto the anterior corneal surface of the eye in a volume of from 25.0 to 200.0 μl, from 25.0 to 100.0 μl, preferably from 50.0 to 100.0 μl, such as about 50.0 μl. The combination of the mold and the limited amount of volume being used further allows avoiding spilling of the crosslinkable liquid composition over the entire anterior eye surface and/or under the eyelids before crosslinking.

In embodiments, the method may comprise maintaining the mold in place on the anterior corneal surface of the eye for the entire period of crosslinking the crosslinkable liquid composition.

In embodiments, if the mold is a contact lens, the center of the contact lens is placed onto the center of the anterior surface of the cornea.

In embodiments of the uses or methods as taught herein, the method may further comprise removing the mold after crosslinking the crosslinkable composition. In embodiments of the methods which involve a correction step, the method may comprise removing the mold after crosslinking the crosslinkable composition and prior to correcting the curvature of the crosslinked composition.

In addition, the present inventors have found that by applying the crosslinkable liquid composition into or onto the corneal stromal surface of the eye by intrastromal injection or after making surgical access, the crosslinkable liquid composition and final crosslinked composition, can be precisely placed onto the corneal stromal surface of the eye and can be shaped into the desired shape. By creating the corneal inlay in situ, it allows correcting a refractive error in a minimally invasive way as no corneal ablation needs to be performed.

Hence, in embodiments of the uses or methods as taught herein, the crosslinking may be performed in situ, onto the corneal stromal surface of the eye. Hence, in other embodiments of the uses or methods as taught herein, the crosslinked composition is a corneal inlay. In embodiments, the crosslinking is performed at body temperature, such as at a temperature of from 35.0° C. to 40.0° C. or from 36.0° C. to 38.0° C.

The term “corneal inlay” generally refers to an optical device positioned into the stromal surface of the cornea.

The wordings “onto a corneal stromal surface” or “onto a stromal surface of the cornea” as used herein includes onto the surface of the corneal stroma and into the corneal stroma.

In embodiments of the uses or methods as taught herein, when the crosslinked composition is a corneal inlay, the method may comprise:
applying the crosslinkable liquid composition onto a corneal stromal surface of the eye comprising the eye disorder,
irradiating the crosslinkable liquid composition on the eye comprising the eye disorder with visible light, thereby obtaining a crosslinked composition, in particular a corneal inlay, on the corneal stromal surface of the eye of the subject, and
cutting a lenticular shape out of the crosslinked composition, in particular the corneal inlay,
wherein the crosslinkable liquid composition is applied onto a corneal stromal surface by intrastromal injection or after making surgical access to the corneal stromal surface.

In embodiments, the crosslinkable liquid composition may be applied onto the corneal stromal surface of the eye by intrastromal injection. In alternative embodiments, the crosslinkable liquid composition may be applied onto the corneal stromal surface of the eye by making surgical access to the corneal stromal surface. The surgical access to the corneal stromal surface of the eye may be created manually, for instance by creating a flap in the corneal stromal surface, or by a laser, e.g. to form a laser-assisted corneal pocket.

In embodiments, the crosslinkable liquid composition may be applied onto the corneal stromal surface of the eye and after its application, the crosslinkable liquid composition may be crosslinked in situ with visible light. Crosslinking may be performed either immediately after introduction of the crosslinkable liquid composition or after closure of the surgical access point.

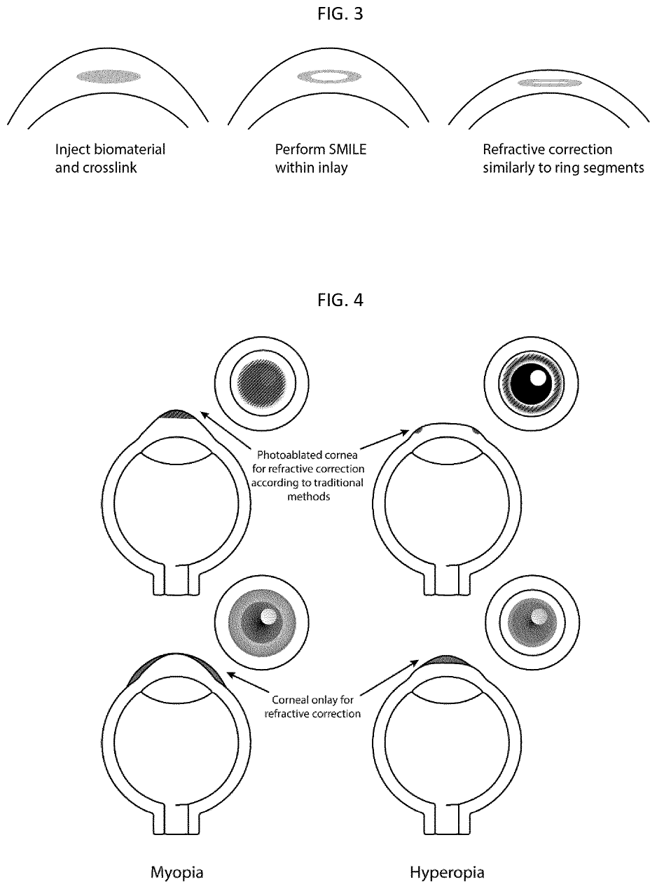

In embodiments, the crosslinkable liquid composition may be injected into the stroma of the eye followed by crosslinking, as illustrated in FIG. 3. Then, in embodiments, a procedure similar to small incision lenticule extraction (SMILE) of the inlay may be performed to allow the formation of an annular inlay and refractive correction similar to placing of a ring segment, as shown in FIG. 3 (and detailed herein below).

In embodiments, the crosslinkable liquid composition may be applied into or onto the corneal stromal surface of the eye in a volume of from 25.0 to 200.0 μl, from 25.0 to 100.0 μl, preferably from 50.0 to 100.0 μl, such as about 50.0 μl.

In embodiments of the uses or methods as taught herein, the crosslinkable liquid composition may be applied as a single layer. In embodiments, the crosslinkable liquid composition may be provided as a single layer onto the anterior corneal surface of the eye. In line therewith, in embodiments, the crosslinked composition on the anterior corneal surface of the eye consists of a single layer of biomaterial. In embodiments, the crosslinkable liquid composition may be provided as a single layer onto the corneal stromal surface of the eye. In line therewith, in embodiments, the crosslinked composition on the corneal stromal surface of the eye consists of a single layer of biomaterial.

In embodiments of the uses and methods as taught herein, prior to applying the crosslinkable liquid composition or the mold onto the anterior corneal surface of the eye, the Optionally, the anterior corneal surface of the eye is debrided, i.e. epithelial cells are removed from the cornea to expose the corneal stromal bed for grafting the corneal onlay thereon. The anterior corneal surface may be debrided of corneal epithelial cells by any means known in the art, such as by use of alcohol delamination, a blunt blade, a diamond burr, a cotton sponge or an Amoils brush. Alternatively, where a mold is used the surgeon can opt to remove the epithelium after placing the mold (to only remove the epithelial cells in that area).

In embodiments, the uses and methods as taught herein may not comprise removal or damaging of the Bowman's layer, the corneal stroma, or a combination thereof prior to applying the crosslinkable liquid composition onto the anterior corneal surface of the eye.

The compositions of the present invention are particularly suitable for procedures which involve crosslinking of the material, more particularly after application on and/or in the eye.

In embodiments, the crosslinking is performed by photocrosslinking by the use of UV or visible light. The term “photocrosslinking” as used herein refers to the process of using electromagnetic radiation, such as visible light, to crosslink compounds such as to crosslink the polymers of the crosslinkable liquid composition and/or to crosslink the polymers of the crosslinkable liquid composition with the corneal surface of the eye. The visible light may be generated by a laser such as a pulsed laser.

In embodiments, photocrosslinking may be laser-assisted photocrosslinking.

The terms “radiation” and “electromagnetic radiation” may be used interchangeably herein.

In embodiments, the electromagnetic radiation is visible light.

In embodiments, the crosslinkable liquid composition is allowed to crosslink until at least 80.0%, preferably at least 90.0%, such as 99.9% or 100.0%, of the crosslinkable liquid composition is crosslinked.

The crosslinkable liquid composition as taught herein advantageously has a fast crosslinking kinetics. This can drastically reduce the crosslinking duration (irradiation time) of the eye, thereby reducing the risks of side-effects and decreasing the overall treatment time.

In embodiments, the crosslinkable liquid composition may be crosslinked using visible or UV light for a period of at least 5 seconds, at least 6 seconds, at least 7 seconds, at least 8 seconds, at least 9 seconds, at least 10 seconds, at least 20 seconds, at least 30 seconds, at least 40 seconds, at least 50 seconds, or at least 60 seconds (1 minute). In embodiments, the crosslinkable liquid composition may be crosslinked using visible or UV light for a period of at most 5 minutes, at most 4 minutes, at most 3 minutes, at most 2 minutes or at most 1 minute. In embodiments, the crosslinkable liquid composition may be crosslinked using visible light for a period of 5 second to 5 minutes, such as for a period of 5 seconds to 4 minutes, 5 seconds to 4 minutes, 5 seconds to 3 minutes, 5 seconds to 2 minutes, or 5 seconds to 1 minute, preferably for a period of 5 seconds to 30 seconds. In embodiments, the UV light is administered with a UV lamp using a dosage of between 1 mW/cm2 and 20 mW/cm2, such as 18 mW/cm2 depending on the nature of the lamp. Examples of suitable UV light sources include the Schwind CXL-365 vario system, OmniCure®1500 and AnalytikJena UV crosslinker. In embodiments, the total dose of UV light may be between 3 and 7J/cm² , such as about 5 J/cm². In particular embodiments the visible light (around 450 nm) is administered with a strength of between 800-1500 mw/cm².

The use or method as taught herein provides a user-friendly and long-term treatment of the refractive error or the chronic or subacute corneal disease involving an irregularity of the cornea. As the crosslinked composition on the anterior corneal surface of the eye is resistant to biodegradation, is less likely to result in post-procedure complications such as dry eye disease, pain or regression of the applied correction, and does not need to be replaced regularly.

In embodiments of the uses or methods as taught herein, the crosslinked composition, such as the corneal onlay or corneal inlay, may be resistant to biodegradation for a period of at least 6 months, preferably for a period of at least 12 months. In embodiments, the crosslinked composition, such as the corneal onlay or corneal inlay, may be resistant to degradation for a period of at least 6 months, preferably at least 12 months. In embodiments, the crosslinked composition, such as the corneal onlay or corneal inlay, may be resistant to enzymatic degradation for a period of at least 6 months, preferably at least 12 months.

In embodiments, the crosslinkable liquid composition, such as the corneal onlay or corneal inlay, may be resistant to degradation by matrix metalloproteinases (MMPs) of the corneal epithelium, such as MMP-1, MMP-2, MMP-3, MMP-9, or a combination thereof.

Crosslinking of the crosslinkable liquid composition onto the corneal surface of the eye leads to adherence between the crosslinked composition and the corneal surface of the eye through the formation of covalent bonds with the amino acids in the corneal collagen. In embodiments, when the crosslinked composition is a corneal onlay, the posterior surface of the crosslinked composition adheres to the Bowman's membrane of the eye with an adhesion strength of at least 10.0 kPa, and preferably with an adhesion strength from 10.0 to 100.0 kPa. In embodiments, when the crosslinked composition is a corneal inlay, the posterior surface of the crosslinked composition adheres to the stroma of the eye with an adhesion strength of at least 10.0 kPa, and preferably with an adhesion strength from 10.0 to 100.0 kPa. The adhesion strength may be determined by any methods known in the art, such as by a lap shear test with a universal testing machine according to the ASTM F2255 using gelatin-coated glass slides.

In embodiments, when the crosslinked composition is a corneal onlay, the posterior surface of the crosslinked composition contacts the Bowman's membrane of the eye. In embodiments, when the crosslinked composition is a corneal inlay, the posterior surface of the crosslinked composition contacts the stroma of the eye.

In embodiments, the crosslinked composition is flexible. The flexibility of the crosslinked composition may be altered by changing the concentration of the functionalized gelatin as taught herein in the crosslinkable liquid composition, its degree of substitution, or its molecular weight. Preferably, the flexibility of the crosslinked composition is similar to native cornea.

In embodiments of the uses or methods as taught herein, the crosslinked composition, such as the corneal onlay or corneal inlay, may have a diameter of from about 6.0 mm to about 9.0 mm and a thickness of from about 10.0 μm to about 400.0 μm prior to correcting the curvature or shape of the crosslinked composition. For instance, the crosslinked composition may have a diameter of from about 6.0 mm to about 8.0 mm and a thickness of from about 10.0 μm to about 400.0 μm prior to correcting the curvature or shape of the crosslinked composition, or the crosslinked composition may have a diameter of from about 7.0 mm to about 9.0 mm and a thickness of from about 10.0 μm to about 400.0 μm prior to correcting the curvature or shape of the crosslinked composition. In particular embodiments, where the curvature or shape of the crosslinked composition does not need to be adjusted or where the correction does not affect the overall thickness of the onlay or inlay, the cross-linked composition will have a thickness of between 10-30 nm, preferably about 15 to 20 μm.

In embodiments, the crosslinked composition may have a diameter of from about 6.0 mm to about 9.0 mm, such as from about 6.0 mm to about 8.0 mm or from about 7.0 mm to about 9.0 mm, prior to correcting the curvature or shape of the crosslinked composition.

In embodiments, the crosslinked composition may have a thickness of from about 10.0 μm to about 400.0 μm, such as from about 20.0 μm to about 400.0 μm, from about 25.0 μm to about 400.0 μm, from about 50.0 μm to about 400.0 μm, from about 100.0 μm to about 400.0 μm, from about 200.0 μm to about 400.0 μm, or from about 100.0 μm to about 300.0 μm, prior to correcting the curvature or shape of the crosslinked composition.

In embodiments, when the crosslinked composition is a corneal onlay, it can be of interest to correct the curvature of the onlay once the crosslinkable composition has been crosslinked onto the anterior corneal surface of the eye. More particularly, the crosslinked composition may be reshaped for optimal and/or patient-specific treatment of the eye disorder, such as a patient-tailored vision correction. For example, the radius of curvature of the crosslinked composition may be corrected, such as by use of photoablation, to treat the eye disorder. Reshaping of the crosslinked composition may change the refractive properties of the so-treated eye in a desired manner to correct the eye disorder, such as the refractive error.

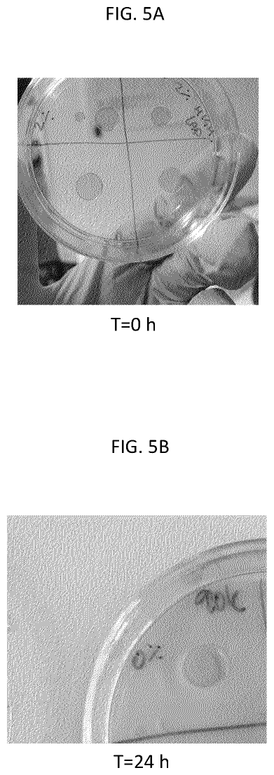

The person skilled in the art will understand that depending on the eye disorder to be treated, the radius of curvature of the crosslinked composition may be corrected differently. For example, for the treatment of farsightedness, biomaterial can be maintained predominantly centrally. Further for example, for the treatment of nearsightedness, biomaterial can be maintained predominantly peripherally. The ablation depth of the corneal onlay is correlated to the envisaged refractive correction and calculated similarly to current refractive laser surgery. Personalized treatment profiles, also known as nomograms, are generated and transferred to a laser for treatment. It is noted that the mode of refractive correction is different from current strategies because of the inherent nature of the corneal onlay technology. Whereas in case of myopia, laser refractive surgery subtracts peripheral tissue, a corneal onlay corrects myopia by adding peripheral tissue. Vice versa, laser refractive surgery corrects hyperopia by tissue dissection mid-peripherally, while a corneal onlay corrects this by adding tissue centrally (FIG. 4).

In embodiments, when the crosslinked composition is a corneal inlay, it can be of interest to correct the shape of the inlay once the crosslinkable composition has been crosslinked onto the corneal stromal surface of the eye. More particularly, the crosslinked composition may be reshaped for optimal and/or patient-specific treatment of the eye disorder, such as a patient-tailored vision correction. For example, the shape of the crosslinked composition may be corrected, such as by use of photoablation, to treat the eye disorder. Reshaping of the crosslinked composition may change the refractive properties of the so-treated eye in a desired manner to correct the eye disorder, such as the refractive error.

For instance, in embodiments, the shape of the crosslinked composition may be altered by cutting a lenticular shape out of the crosslinked composition to create an annular or ring-shaped inlay. The annular inlay may change the refractive properties of the so-treated eye in a desired manner to correct the eye disorder, such as the refractive error, in a manner similar to ring segments. In embodiments, a lenticular shape (lenticule) or an annular shape may be cut out of the crosslinked material by using a laser. The laser may also make an incision on the corneal surface, e.g. an access incision, that is less than 4 mm wide. The lenticule may be removed out of the eye through the access incision, thereby changing the shape of the cornea and correcting the refractive error. The access incision is small and may easily heal. Preferably, where material is removed, a minimal layer of hydrogel is maintained on the eye, such as a layer of 5 μm or more.

In embodiments, the crosslinked composition may be photoablated to obtain a refractive correction, preferably a spherical refractive correction, in the range of from −20 diopters to +10 diopters. In embodiments, the crosslinked composition may be photoablated such that the crosslinked composition comprises at least a central portion having a substantially uniform thickness extending from the lower surface to the upper surface of the crosslinked composition such that the crosslinked composition has an optical power within a range from −20 diopters to about +10 diopters, preferably from −10 diopters to about +5 diopters, along at least the inner portion of the crosslinked composition. In embodiments, the crosslinked composition may be photoablated to obtain a non-spherical shape when the eye disorder is astigmatism. In particular embodiments, manual (by blade) shaping of the onlay may also be envisaged.

In certain embodiments, particularly where the eye-disorder is not a refractive error but rather an irregularity of the cornea, the crosslinked composition may be adjusted to reproduce a natural surface, with minimal impact on vision.

In embodiments, the crosslinked composition may be photoablated to obtain a lens-shape, such as having a thickness of from 10.0 μm to 50.0 μm at the periphery of the outer portion of the crosslinked composition and extending to the central portion of the crosslinked composition with an increasing thickness to from 30.0 μm to 100.0 μm.

In embodiments, the crosslinked composition may be photoablated to obtain a ring-shape.

In embodiments, the upper surface of the crosslinked biomaterial may be photoablated to shape the upper surface.

Furthermore, the person skilled in the art will also understand that the possible deswelling of the crosslinked composition upon overgrowth of the epithelial cells should be taken into account when determining the amount of crosslinked composition that will be removed from the eye to treat the eye disorder.

Photoablation may be performed using a laser, such as an excimer laser or solid-state laser.

In embodiments, the correcting of the curvature of the crosslinked composition may not comprise removing corneal tissue, such as corneal stromal tissue, such as by photoablation.

In particular embodiments, the methods do not involve correction of the surface of the corneal onlay.

In embodiments, no correction of the curvature or shape of the crosslinked material is necessary. This can be the case where the crosslinkable liquid composition is injected into the stroma, where the layer of material is very thin and/or where a mold can be used which ensures exactly the desired shape and thickness of the material after crosslinking. In embodiments, photoablation of the corneal onlay may not be performed when the principal aim is to treat chronic or subacute corneal disease.

In embodiments, the method as taught herein is reversible, meaning that the crosslinked composition may be completely removed from the anterior corneal surface or the corneal stromal surface of the eye, if needed, such as by photoablation, hydrodissection, microkeratome or manual dissection.

After correcting the curvature or shape of the crosslinked composition, the corneal epithelium may spontaneously reform originating from the corneal limbus, the corneal scleral transition zone. Overgrow of the crosslinked composition by the corneal epithelium typically occurs within 1 to 2 weeks after correcting the curvature or shape of the crosslinked composition. In embodiments, one or more therapeutic agents, such as NGF, may be administered to the eye to improve regrowth of the corneal epithelium.

The subject may post-operatively be treated with therapeutic agents that reduce pain and/or inflammation, such as corticosteroids and/or antibiotics.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as follows in the spirit and scope of the appended claims.

The herein disclosed aspects and embodiments of the invention are further supported by the following non-limiting examples.

### EXAMPLES

### Example 1: Ultraviolet (UV) and High-Energy Visible (HEV) Light Blocking Properties of a Crosslinked Composition Obtained from a Crosslinkable Liquid Composition for Use in a Method of Treating an Eye Disorder in an Eye of a Subject According to Embodiments of the Invention

The following materials were used: Eppendorf tubes amber colored (VWR, 525-1223), Gelatin methacrylate DS90 or DS160 (Rousselot), Lithium phenyl-2,4,6-trimethylbenzoylphosphinate (Merck, 900889-1G), Poly ethylene glycol diacrylate (Merck, 907227-1G), Gelatin desaminotyrosine (GelDAT) (Rousselot), Photoinitiator kit including ruthenium and SPS (Merck, 916811-1EA), Phosphate buffered saline (Thermo Fisher), CCL-365 UV lamp (Vario) or CL-3000 UV crosslinker (AnalytikJena) and a Spark Cyto spectrophotometer (Tecan).

Hydrogel formulations were prepared in eppendorf tubes according to the desired concentrations of functionalised gelatin (in the desired molecular weight), photoinitiator and co-initiator.

In a first analysis a sample was prepared as follows: 20% 90 kDa GelDAT, 1 mM/Rut, 10 mM SPS (volume: 50 μL);
Droplets of 50 μL were placed inside the largest lid of a petri dish. The small lid was placed upside down on the droplets to press them down. The hydrogels were crosslinked by irradiating them with CCL-365 UV lamp for 5 minutes at 18 mW/cm² or with a CL-3000 UV crosslinker (AnalytikJena) at 1 mW/cm² to obtain an accumulated UV dosage of 5.4 J/cm².

Crosslinked hydrogels appear most yellow immediately after crosslinking (FIG. 5A, T=0 h), while less intense yellow/orange appearance is noticed after 24 h of washing (FIG. 5B, T=24 h).

The samples were then fully hydrated and placed in 24 well plates with a highly transmissive bottom. The transmission of the samples was recorded over a spectrum of 350-800 nm (Spark Cyto, Tecan instruments) with phosphate buffered saline (PBS) as a reference.

Absorbance values were then converted to the percentage of light transmittance with the following equation (assuming that there is little to no reflection):

$$A = 2 - log \% T \to \% T = {10}^{( 2 - A )}$$

wherein A is absorbance and % T is the percentage of transmittance.

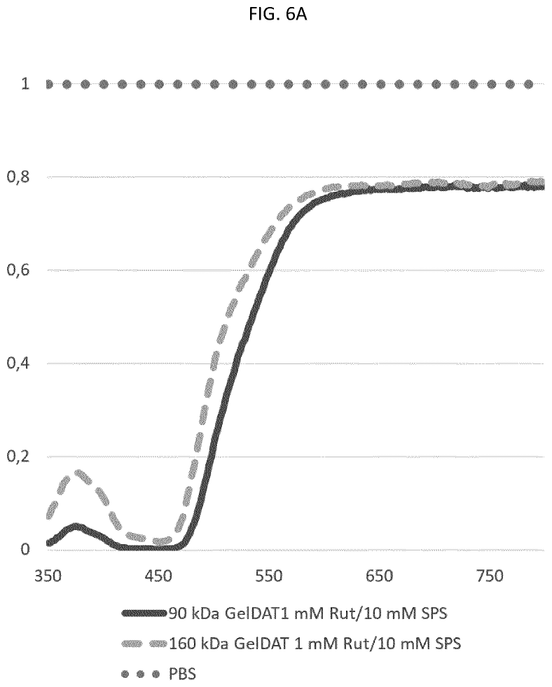

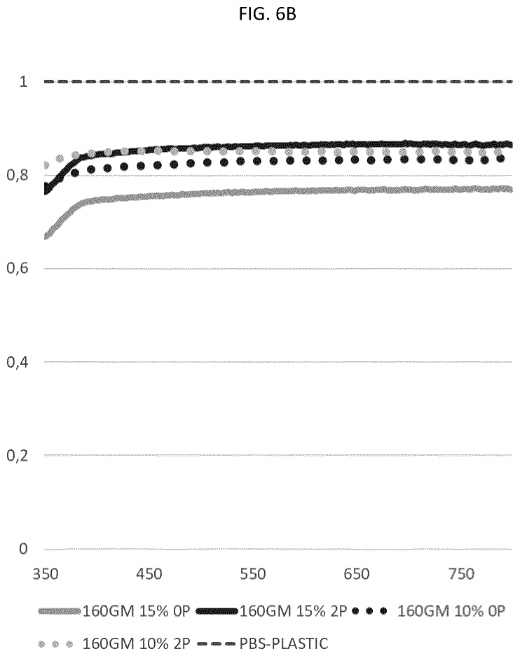

The results are shown in FIG. 6.

Samples FIG. 6A:
10% GelDAT 90 kDa 1 mM RUT/10 mM SPS (solvent: PBS)
10% GelDAT 160 kDa 1 mM RUT/10 mM SPS (solvent: PBS)
Phosphate buffered saline (control)

Samples FIG. 6B:
10% GelMA 160 kDa-0% PEGDA (solvent: PBS)
10% GelMA 160 kDa-2% PEGDA (0.0625% LAP) (solvent: PBS)
15% GelMA 160 kDa-0% PEGDA (solvent: PBS)
15% GelMA 160 kDa-2% PEGDA (0.0625% LAP)
(solvent: PBS) Phosphate buffered saline (control)

FIG. 6: The transmittance of the compositions of the invention (i.e., with a UV or visible light excitable photoinitiator such as ruthenium) were compared to a comparative crosslinked composition obtained with a UV excitable photoinitiator (i.e., Lithium phenyl-2,4,6-trimethylbenzoylphosphinate, abbreviated as LAP) that does not give rise to a yellow appearance. In FIG. 6A the UV and visible light filtering hydrogel was not transmitting light between at 350-470 nm. On the other hand, in FIG. 6B, the hydrogel crosslinked with normal UV light photoinitiators (i.e., LAP) is not blocking light in the spectrum of 350-800 nm. As a positive control and reference value phosphate buffered saline was measured.

### Example 2: Crosslinking Kinetics of a Crosslinked Composition Obtained from a Crosslinkable Liquid Composition for Use in a Method of Treating an Eye Disorder in an Eye of a Subject According to Embodiments of the Invention

The following materials were used: Eppendorf tubes amber colored (VWR, 525-1223); Gelatin methacrylate DS90 or DS160 (Rousselot); Lithium phenyl-2,4,6-trimethylbenzoylphosphinate (Merck, 900889-1G); Gelatin desaminotyrosine (GelDAT) (Rousselot); Photoinitiator kit including ruthenium and SPS (Merck, 916811-1EA); Phosphate buffered saline (Thermo Fisher); Anton Paar MCR302e rheometer; Omnicure s1500 crosslinking lamp.

The hydrogel formulations were prepared in eppendorf tubes according to the desired concentrations of functionalised gelatin (in the desired molecular weight), photoinitiator and co-initiator (not for methacrylated samples). The following samples were prepared:
Sample 1: 20% GelMA 90 kDa 0.0625% LAP (solvent: PBS)
Sample 2: 20% GelMA 160 kDa 0.0625% LAP (solvent: PBS)
Sample 3: 20% GelDAT 90 kDa 1 mM Rut/10 mM SPS (solvent: PBS)
Sample 4: 15% GelDAT 160 kDa 1 mM Rut/10 mM SPS (solvent: PBS)

The fluid crosslinkable compositions were each placed in the rheometer (Anton Paar MCR302e) with a coupled Omnicure s1500 crosslinking lamp. The gap was set at 0.5 mm with an irradiation protocol of 60 seconds of measurement without UV, followed by 5 minutes of UV light. The G′ and G″ values were measured every 10 seconds and plotted over time. The increasing storage modulus reflects the crosslinking process as the hydrogel becomes stiffer upon polymerization.

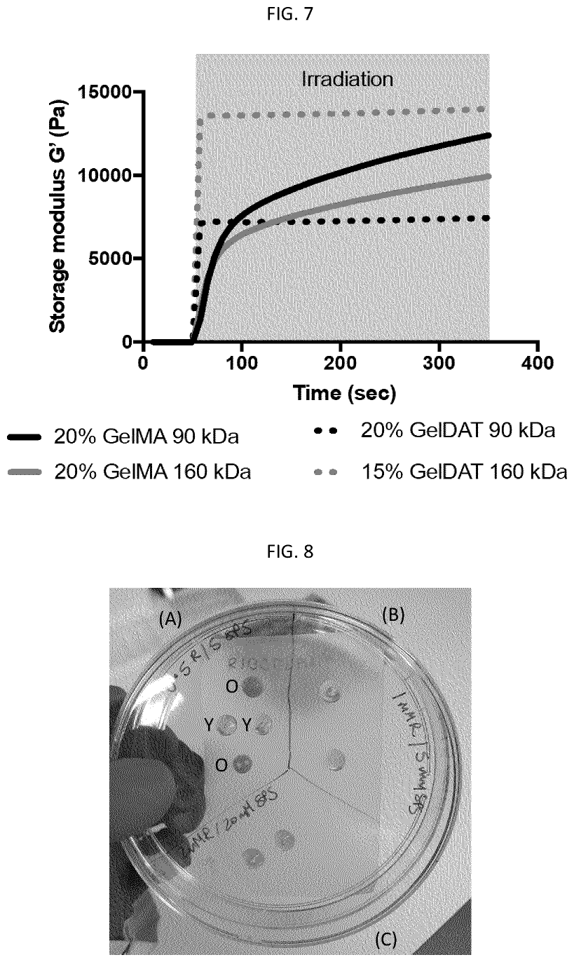

The results are provided in FIG. 7. The full lines represent comparative crosslinked composition comprising GelMA and polymerized with a UV based photoinitiator. In the first 30 seconds of irradiation, the storage modulus increased exponentially following a moderate increase until t=350 seconds (FIG. 7, full lines). These hydrogels did not crosslink 100% during this time span as the storage modulus did not reach a stable plateau phase (FIG. 7, full lines). In contrast, the crosslinked composition illustrating the invention (FIG. 7, dashed lines) reached a stable plateau already on the first time point after irradiation, indicating their very fast crosslinking kinetics versus the UV based crosslinking system.

It can be seen that an additional advantage of using the crosslinkable liquid compositions illustrating the invention is their very fast crosslinking kinetics. This can drastically reduce the crosslinking duration (irradiation time) of the eye, thereby reducing the risks of side-effects and decreasing the overall treatment time.

### Example 3: Exemplary Method of Treating an Irregularity of the Cornea with the Compositions of the Invention (such as in FIGS. 1 and 2)

The irregularity of the cornea is diagnosed according to an established method.

After preparing the patient for the procedure, the patient's corneal epithelium is removed using diluted alcohol.

The ring of a vacuum suction device is placed on top of the cornea of the patient. About 50 μl of the uncrosslinked polymer (20% GelDAT 90 kDa 1 mM Rut/10 mM SPS) is applied in the vacuum suction device.

The treated eye is irradiated to fully crosslink and adhere the corneal onlay with about e.g. 5.4 J/cm2 of ultraviolet light of 365 nm. After the crosslinking the ring of the suction device is removed.

The polymer covalently adheres to extracellular matrix of the eye during the crosslinking process and is allowed to rehydrate (isotonic eye drops can be administered).

The crosslinked material is allowed to equilibrate for a period of about 4 hours.

Laser refractive surgery is then performed on the corneal onlay (and not the cornea tissue) thereby correcting the curvature of the newly formed corneal onlay.

### Example 4: UV and HEV Light Blocking Properties of a Crosslinked Composition Obtained from a Crosslinkable Liquid Composition Comprising GelDAT and Riboflavin for Use in a Method of Treating an Eye Disorder in an Eye of a Subject According to Embodiments of the Invention

The following materials were used: Eppendorf tubes amber colored (VWR, 525-1223), Gelatin desaminotyrosine (GelDAT) 90 kDa (Rousselot), Riboflavin (Merck, R9504-25G), Sodium persulphate (SPS) (Merck, 916811-1EA), Phosphate buffered saline 1× (Thermo Fisher, 14200083), UV 15 crosslinker CL-3000 (AnalytikJena, CL-3000).

GelDAT, Ruthenium and SPS were prepared in separate amber Eppendorfs as follows:
15% (w/v) GelDAT: PBS 1× was added to GelDAT powder at a ratio of 0.075 g powder: 0.5 ml PBS. The vial was placed on a rocker in an incubator at 37° C. until the powder completely dissolved and gel formed an even consistency.
riboflavin stock solutions were made by shaking on rocker for at least 1 hour or until powder completely dissolved.
SPS stock solutions were prepared by placing on rocker for at least 1 hour or until powder completely dissolved.
Liquid components were mixed in a fresh amber Eppendorf, to the required concentration/volume and labeled with date, contents and initials: (A) 15% GelDAT/0.5 mM Riboflavin/5 mM SPS, (B) 15% GelDAT/1 mM Riboflavin/5 mM SPS, (C) 15% GelDAT/2 mM Riboflavin/20 mM SPS.

Droplets (50 μl) were pipetted into a parafilm-lined petri dish. The petri-dish was closed with the lid.

The petri dish was placed in the UV crosslinker (AnalytikJena) at 1 mW/cm2 to obtain an accumulated UV dosage of 5.4 J/cm2 (time for full crosslinking: 20 minutes). Alternatively, the CCL-365 UV lamp can be used for 5 minutes at 18 mW/cm2, although only one droplet at once can be irradiated.

The crosslinked compositions appeared yellow (Y) or orange (O) immediately after crosslinking for the crosslinkable liquid composition comprising 15% GelDAT/0.5 mM Riboflavin/5 mM SPS (FIG. 8A) or appeared yellow for the crosslinkable liquid composition comprising 15% GelDAT/1 mM Riboflavin/5 mM SPS or for the crosslinkable liquid composition comprising 15% GelDAT/2 mM Riboflavin/20 mM SPS (FIGS. 8B and 8C).

In order to test the UV and HEV light blocking properties, crosslinked composition were prepared as described above from crosslinkable liquid compositions comprising 15% (w/v) 120 kDa GelDAT 50 DoM (50% degree of modification) (Rousselot, X-Pure GelDAT 120P50RG); 0.4 mM (striped line), 0.8 mM (doted and striped line), or 1.4 mM (dotted line) riboflavin (Merck, R 9504-25G); and 4.5 mM, 8 mM and 14 mM SPS, respectively (Merck, 916811-1EA).

After crosslinking, the crosslinked compositions were fully hydrated and placed in 24 well plates with a highly transmissive bottom. The transmission of the samples was recorded over a spectrum of 350-800 nm (Spark Cyto, Tecan instruments) with phosphate buffered saline (PBS) as a reference.

Absorbance values were then converted to the percentage of light transmittance with the following equation (assuming that there is little to no reflection):

$$A = 2 - log \% T \to \% T = {10}^{( 2 - A )}$$

wherein A is absorbance and % T is the percentage of transmittance.

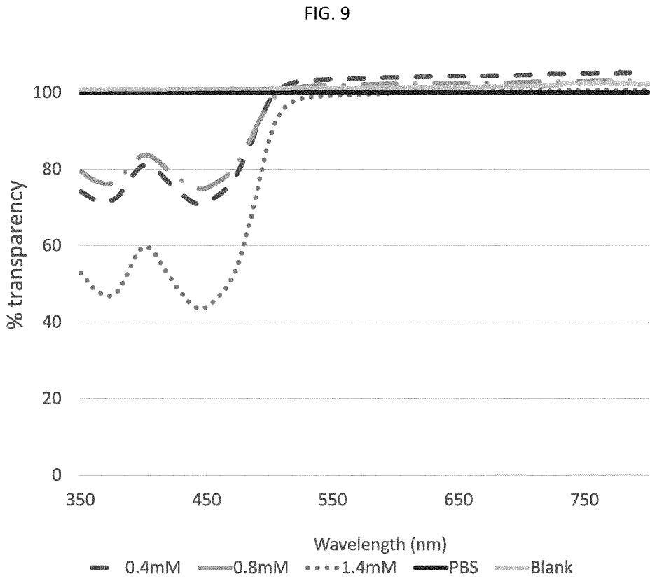

The results are shown in FIG. 9. FIG. 9 illustrates that the compositions of the invention with a photoinitiator such as riboflavin were partially blocking light between 300 nm and 480 nm. When using riboflavin instead of ruthenium as a crosslinking system (FIG. 6A), the crosslinked composition became partly less permeable to light between 300 and 480 nm depending on the concentration of the riboflavin. The effect was most pronounced at 1.4 mM riboflavin but it was not as pronounced as for a crosslinked composition comprising GelDAT and ruthenium for which (almost) complete light blocking effect was obtained between 350 nm and 470 nm (FIG. 6A).

1.-17. (canceled)

18. A method of curative or preventive treatment of an eye disorder in an eye of a subject in need thereof, comprising administering a crosslinkable liquid composition comprising:
a crosslinkable biomaterial, wherein the crosslinkable biomaterial comprises a gelatin functionalized with one or more tyrosine-derived or phenol groups; and
a visible or UV light photoinitiator system,
to the eye of the subject.

19. The method according to claim 18, wherein the method comprises:
applying the crosslinkable liquid composition onto an anterior corneal surface or onto a corneal stromal surface of the eye comprising the eye disorder,
irradiating the crosslinkable liquid composition on the eye comprising the eye disorder with visible or UV light, thereby obtaining a crosslinked composition on the anterior corneal surface or on the corneal stromal surface of the eye of the subject, and
reshaping the crosslinked composition.

20. The method according to claim 18, wherein the method comprises:
applying the crosslinkable liquid composition onto an anterior corneal surface of the eye comprising the eye disorder,
irradiating the crosslinkable liquid composition on the eye comprising the eye disorder with visible or UV light, thereby obtaining a crosslinked composition on the anterior corneal surface of the eye of the subject, and optionally
correcting the curvature of the crosslinked composition,
wherein the method comprises introducing the crosslinkable liquid composition into a mold which is positioned onto the anterior corneal surface of the eye;
or wherein the method comprises:
applying the crosslinkable liquid composition onto a corneal stromal surface of the eye comprising the eye disorder,
irradiating the crosslinkable liquid composition on the eye comprising the eye disorder with visible or UV light, thereby obtaining a crosslinked composition on the corneal stromal surface of the eye of the subject, and optionally
cutting a lenticular shape out of the crosslinked composition,
wherein the crosslinkable liquid composition is applied onto a corneal stromal surface by intrastromal injection or after making surgical access to the corneal stromal surface.

21. The method according to claim 18, wherein the eye disorder is cataract, macular degeneration, a corneal refractive disorder, or a chronic or subacute corneal disease involving an irregularity of the cornea.

22. The method according to claim 21, wherein the corneal refractive disorder is selected from myopia, hyperopia, regular and irregular astigmatism, and presbyopia.

23. The method according to claim 18, wherein the eye disorder is cataract, macular degeneration, a corneal refractive error, or a combination thereof.

24. The method according to claim 23, wherein the eye disorder is cataract, macular degeneration, myopia, hyperopia, astigmatism, presbyopia, or a combination thereof.

25. The method according to claim 21, wherein the chronic or subacute corneal disease involving an irregularity of the cornea is selected from corneal ulcer, corneal erosion, a corneal ectasia, and a corneal irregularity caused by trauma or epithelial basement membrane disorder.

26. The method according to claim 25, wherein the corneal ectasia is keratoconus.

27. The method according to claim 18, wherein the visible or UV light photoinitiator system comprises a photoinitiator and a co-initiator, wherein the photoinitiator comprises tris(2,2′-bipyridine)ruthenium(II) or riboflavin, and the co-initiator is sodium persulphate.

28. The method according to claim 27, wherein the photoinitiator is tris(2,2′-bipyridyl)dichlororuthenium(II) hexahydrate or riboflavin, and the co-initiator is sodium persulphate.

29. The method according to claim 18, wherein the visible or UV light photoinitiator system comprises a photoinitiator and a co-initiator, wherein the photoinitiator comprises tris(2,2′-bipyridine)ruthenium(II) and the co-initiator is sodium persulphate.

30. The method according to claim 29, wherein the photoinitiator is tris(2,2′-bipyridyl)dichlororuthenium(II) hexahydrate and the co-initiator is sodium persulphate.

31. The method to claim 18, wherein the gelatin functionalized with one or more tyrosine-derived or phenol groups is a gelatin desaminotyrosine.

32. The method according to claim 20, wherein the mold is a corneal vacuum suction device, a corneal bath or a contact lens.

33. The method according to claim 20, wherein the method comprises applying the mold onto the anterior corneal surface of the eye either before or after applying said crosslinkable liquid composition into the mold.

34. The method according to claim 20, wherein the method further comprises removing the mold after crosslinking the crosslinkable composition.

35. The method according to claim 18, wherein the crosslinkable liquid composition is applied as a single layer.

36. The method according to claim 18, wherein the crosslinked composition is a corneal onlay or a corneal inlay.

37. The method according to claim 18, wherein the crosslinked composition has a diameter of from 6.0 mm to 9.0 mm and a thickness of from 10.0 μm to 400.0 μm prior to correcting the curvature or shape of the crosslinked composition; and/or wherein the crosslinked composition is resistant to biodegradation for a period of at least 6 months.

38. A surgical method of preparing a corneal onlay on or a corneal inlay in an eye of a subject in need thereof, comprising administering a crosslinkable liquid composition comprising:
a crosslinkable biomaterial, wherein the crosslinkable biomaterial comprises a gelatin functionalized with one or more tyrosine-derived or phenol groups; and
a visible and/or UV light photoinitiator system,
to the eye of the subject.

39. The method according to claim 38, wherein the method comprises:
applying the crosslinkable liquid composition onto an anterior corneal surface or onto a corneal stromal surface of the eye comprising the eye disorder,
irradiating the crosslinkable liquid composition on the eye comprising the eye disorder with visible or UV light, thereby obtaining a corneal onlay on the anterior corneal surface or a corneal inlay on the corneal stromal surface of the eye of the subject, and
reshaping the crosslinked composition.

40. The method according to claim 37, wherein the method comprises:
applying the crosslinkable liquid composition onto an anterior corneal surface of the eye comprising the eye disorder,
irradiating the crosslinkable liquid composition on the eye comprising the eye disorder with visible and/or UV light, thereby obtaining a corneal onlay on the anterior corneal surface of the eye of the subject, and optionally
correcting the curvature of the corneal onlay,
wherein the method comprises introducing the crosslinkable liquid composition into a mold which is positioned onto the anterior corneal surface of the eye;
or wherein the method comprises:
applying the crosslinkable liquid composition onto a corneal stromal surface of the eye comprising the eye disorder,
irradiating the crosslinkable liquid composition on the eye comprising the eye disorder with visible and/or UV light, thereby obtaining a corneal inlay on the corneal stromal surface of the eye of the subject, and optionally
cutting a lenticular shape out of the corneal inlay,
wherein the crosslinkable liquid composition is applied onto a corneal stromal surface by intrastromal injection or after making surgical access to the corneal stromal surface.
